(19) United States
(12) Patent Application Publication
(10) Pub. No.: US 2012/0235123 A1
(43) Pub. Date: Sep. 20, 2012

(54) NOVEL ORGANIC ELECTROLUMINESCENT COMPOUNDS AND ORGANIC ELECTROLUMINESCENT DEVICE USING THE SAME

(75) Inventors: Soo Young Lee, Namyangju-si (KR); Young Jun Cho, Seongbuk-gu (KR); Hyuck Joo Kwon, Dongdaemun-gu (KR); Bong Ok Kim, Gangnam-gu (KR); Sung Min Kim, Yangcheon-gu (KR); Seung Soo Yoon, Suwon-si (KR)

(73) Assignee: ROHM AND HAAS ELECTRONIC MATERIALS KOREA LTD., Chungcheon-do (KR)

(21) Appl. No.: 13/389,750

(22) PCT Filed: Aug. 3, 2010
(86) PCT No.: PCT/KR2010/005092

§ 371 Date: Apr. 27, 2012

(30) Foreign Application Priority Data
Aug. 10, 2009 (KR) 1020090073260

(51) Int. Cl. H01L 51/54; C07F 9/6568; C07D 405/14

(52) U.S. Cl. 257/40; 546/276.7; 546/143; 546/159; 544/331; 544/216; 544/214; 257/E51.026

(57) ABSTRACT

Provided are a novel organic electroluminescent compound and an organic electroluminescent device using the same. More particularly, the organic electroluminescent compound disclosed herein is represented by Chemical Formula 1:

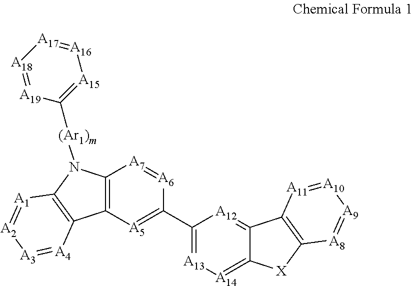

Since the organic electroluminescent compound disclosed herein exhibits good luminous efficiency and excellent life property, it may be used to manufacture OLED devices having very superior operation life.

### FIELD OF THE INVENTION

The present invention relates to novel organic electroluminescent compounds and an organic electroluminescent device using the same. The organic electroluminescent compound according to the present invention is represented by Chemical Formula 1:

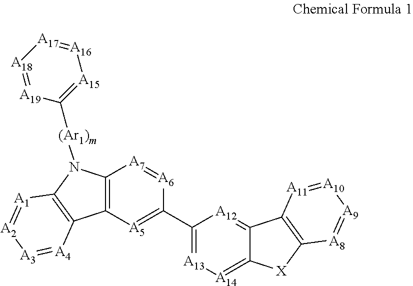

### BACKGROUND OF THE INVENTION

In general, the organic EL device commonly has a configuration of anode/hole injection layer (HIL)/hole transport layer (HTL)/emission material layer (EML)/electron transport layer (ETL)/electron injection layer (EIL)/cathode. Organic electroluminescent devices emitting blue, green or red light may be created depending on how to form the emission material layer.

At present, 4,4′-bis(carbazol-9-yl)biphenyl (CBP) is the most widely known as a host material for a phosphorescent material. High-efficiency OLEDs using a hole blocking layer comprising 2,9-dimethyl-4,7-diphenyl-1,10-phenanthroline (BCP), Bis(2-methyl-8-quinolinato)(p-phenyl-phenolato)aluminum(III) (BAlq), etc. are reported. High-performance OLEDs using BAlq derivatives as a host were reported by Pioneer (Japan) and others.

Although these materials provide good electroluminescence characteristics, they are disadvantageous in that degradation may occur during the high-temperature deposition process in vacuum because of low glass transition temperature and poor thermal stability. Since the power efficiency of an OLED is given by (π/voltage)×current efficiency, the power efficiency is inversely proportional to the voltage. High power efficiency is required to reduce the power consumption of an OLED. Actually, OLEDs using phosphorescent materials provide much better current efficiency (cd/A) than those using fluorescent materials. However, when the existing materials such as BAlq, CBP, etc. are used as a host of the phosphorescent material, there is no significant advantage in power efficiency (lm/W) over the OLEDs using fluorescent materials because of high driving voltage.

Further, the OLED devices do not have satisfactory operation life. Therefore, development of more stable, higher-performance host materials is required.

### TECHNICAL PROBLEM

Accordingly, an object of the present invention is to provide an organic electroluminescent compound having luminescence efficiency and device operation life improved over existing host materials and having superior backbone with appropriate color coordinates in order to solve the aforesaid problems. Another object of the present invention is to provide a highly efficient and long-life organic electroluminescent device employing the organic electroluminescent compound as an electroluminescent material.

### TECHNICAL SOLUTION

The present invention relates to organic electroluminescent compounds represented by Chemical Formula 1, and an organic electroluminescent device using the same. The organic electroluminescent compounds according to the present invention exhibit high luminous efficiency, excellent color purity and life property of the material, so that OLED's with very excellent operation life can be manufactured therefrom.

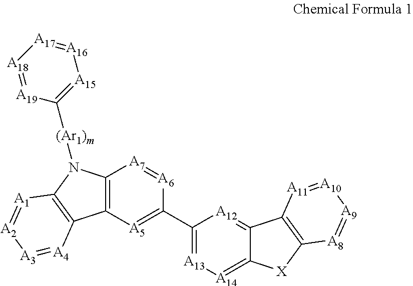

wherein

A₁ through A₁₉ independently represent CR₁ or N, X represents —(CR₂R₃)₁—, —N(R₄)—, —S—, —O—, —Si (R₅)(R₆)—, —P(R₇)—, —P(═O)(R₈)— or —B(R₉)—, and Ar₁ represents (C6-C40)arylene with or without substituent(s) or (C3-C40)heteroarylene with or without substituent(s), except for the case where m is 0, and A₁₅ through A₁₉ are CR₁ at the same time;

R₁ through R₉ independently represent hydrogen, deuterium, halogen, (C1-C30)alkyl with or without substituent(s), (C6-C30)aryl with or without substituent(s), substituted or unsubstituted (C6-C30)aryl fused with one or more (C3-C30)cycloalkyl(s) with or without substituent(s), (C3-C30)heteroaryl with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), cyano, trifluoromethyl, NR₂₁R₂₂, BR₂₃R₂₄, PR₂₅R₂₆, P(═O)R₂₇R₂₈, R^{a}R^{b}R^{c}Si—, R^{d}Y—, R^{e}C(═O)—, R^{f}C(═O)O—, (C6-C30)ar(C1-C30)alkyl with or without substituent(s), (C2-C30)alkenyl with or without substituent(s), (C2-C30)alkynyl with or without substituent(s), carboxyl, nitro,

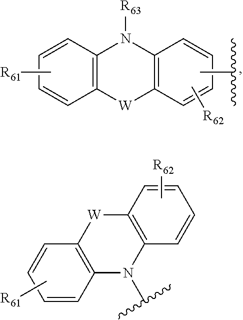

or hydroxyl, or each of them may be linked to an adjacent substituent via (C3-C30)alkylene or (C3-C30)alkenylene with or without a fused ring to form an alicylic ring, a mono- or polycyclic aromatic ring or a mono- or polycyclic heteroaromatic ring;

W represents —(CR₅₁R₅₂)ₙ—, —(R₅₁)C═C(R₅₂)—, —N(R₅₃)—, —S—, —O—, —Si(R₅₄)(R₅₅)—, —P(R₅₆)—, —P(═O)(R₅₇)—, —C(═O)— or —B(R₅₈)—, and R₅₁ through R₅₈ and R₆₁ through R₆₃ are the same as R₁ through R₉;

the heterocycloalkyl or heteroaryl may contain one or more heteroatom(s) selected from B, N, O, S, P(═O), Si and P;

R₂₁ through R₂₈ independently represent (C1-C30)alkyl with or without substituent(s), (C6-C30)aryl with or without substituent(s) or (C3-C30)heteroaryl with or without substituent(s), R^{a}, R^{b}, and R^{c} independently represent (C1-C30)alkyl with or without substituent(s) or (C6-C30)aryl with or without substituent(s), Y represents S or O, R^{d} represent (C1-C30)alkyl with or without substituent(s) or (C6-C30)aryl with or without substituent(s), R^{e} represent (C1-C30)alkyl with or without substituent(s), (C1-C30)alkoxy with or without substituent(s), (C6-C30)aryl with or without substituent(s) or (C6-C30)aryloxy with or without substituent(s), R^{f} represent (C1-C30)alkyl with or without substituent(s), (C1-C30)alkoxy with or without substituent(s), (C6-C30)aryl with or without substituent(s) or (C6-C30)aryloxy with or without substituent(s);

m represents an integer 0 to 2; and

l and n represent an integer 1 or 2.

In the present invention, “alkyl”, “alkoxy” and other substituents containing “alkyl” moiety include both linear and branched species. In the present invention, “cycloalkyl” includes both adamantyl with or without substituent(s) and (C7-C30)bicycloalkyl with or without substituent(s).

In the present invention, “aryl” means an organic radical derived from an aromatic hydrocarbon by the removal of one hydrogen atom, and may include a 4- to 7-membered, particularly 5- or 6-membered, single ring or fused ring, including a plurality of aryls linked by chemical bond(s). Specific examples include phenyl, naphthyl, biphenyl, anthryl, indenyl, fluorenyl, phenanthryl, triphenylenyl, pyrenyl, perylenyl, chrysenyl, naphthacenyl, fluoranthenyl, etc., but are not limited thereto. “heteroaryl” means an aryl group containing 1 to 4 heteroatom(s) selected from B, N, O, S, P(═O), Si and P as aromatic ring backbone atom(s), other remaining aromatic ring backbone atoms being carbon. It may be 5- or 6-membered monocyclic heteroaryl or polycyclic heteroaryl resulting from condensation with a benzene ring, and may be partially saturated. Further, the heteroaryl includes more than one heteroaryls linked by chemical bond(s). The heteroaryl includes a divalent aryl group wherein the heteroatom(s) in the ring may be oxidized or quaternized to form, for example, an N-oxide or a quaternary salt.

Specific examples include monocyclic heteroaryl such as furyl, thienyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., polycyclic heteroaryl such as benzofuryl, benzothienyl, isobenzofuryl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinolizinyl, quinoxalinyl, carbazolyl, phenanthridinyl, benzodioxolyl, etc., an N-oxide thereof (e.g., pyridyl N-oxide, quinolyl N-oxide, etc.), a quaternary salt thereof, etc., but are not limited thereto.

The “(C1-C30)alkyl” groups described herein may include (C1-C20)alkyl or (C1-C10)alkyl and the “(C6-C30)aryl” groups include (C6-C20)aryl or (C6-C12)aryl. The “(C3-C30)heteroaryl” groups include (C3-C20)heteroaryl or (C3-C12)heteroaryl and the “(C3-C30)cycloalkyl” groups include (C3-C20)cycloalkyl or (C3-C7)cycloalkyl. The “(C2-C30)alkenyl or alkynyl” group include (C2-C20)alkenyl or alkynyl, (C2-C10)alkenyl or alkynyl.

In “with or without substituent(s)”, the substituent is further substituted by one or more substituent(s) selected from the group consisting of deuterium, halogen, (C1-C30)alkyl with or without halogen substituent(s), (C6-C30)aryl, (C3-C30)heteroaryl with or without (C6-C30)aryl substituent(s), 5- to 7-membered heterocycloalkyl, 5- to 7-membered heterocycloalkyl fused with one or more aromatic ring(s), (C3-C30)cycloalkyl, (C3-C30)cycloalkyl fused with one or more aromatic ring(s), tri(C1-C30)alkylsilyl, di(C1-C30)alkyl(C6-C30)arylsilyl, tri(C6-C30)arylsilyl, (C2-C30)alkenyl, (C2-C30)alkynyl, cyano, carbazolyl, NR₃₁R₃₂, BR₃₃R₃₄, PR₃₅R₃₆, P(═O)R₃₇R₃₈, (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro and hydroxyl, or is linked to an adjacent substituent to form a ring, wherein R₃₁ through R₃₈ independently represent (C1-C30)alkyl, (C6-C30)aryl or (C3-C30)heteroaryl.

The R₁ through R₉, R₂₁ through R₂₈, R₅₁ through R₅₈ and R₆₁ through R₆₃ are independently selected from hydrogen, deuterium, halogen, alkyl such as methyl, ethyl, propyl, butyl, pentyl, hexyl, ethylhexyl, heptyl, octyl, etc., aryl such as phenyl, naphthyl, fluorenyl, biphenyl, phenanthryl, terphenyl, pyrenyl, perylenyl, spirobifluorenyl, fluoranthenyl, chrysenyl, triphenylenyl, etc., aryl fused with one or more cycloalkyl such as 1,2-dihydroacenaphthyl, heteroaryl such as dibenzothiophenyl, dibenzofuryl, carbazolyl, pyridyl, furyl, thienyl, quinolyl, triazinyl, pyrimidinyl, pyridazinyl, quinoxalinyl, phenanthrolinyl, etc., heterocycloalkyl fused with one or more aromatic ring such as benzopyrrolidino, benzopiperidino, dibenzomorpholino, dibenzoazepino, etc., amino substituted by aryl such as phenyl, naphthyl, fluorenyl, biphenyl, phenanthryl, terphenyl, pyrenyl, perylenyl, spirobifluorenyl, fluoranthenyl, chrysenyl, triphenylenyl, etc. or heteroaryl such as dibenzothiophenyl, dibenzofuryl, carbazolyl, pyridyl, furyl, thienyl, quinolyl, triazinyl, pyrimidinyl, pyridazinyl, quinoxalinyl, phenanthrolinyl, etc., aryloxy such as biphenyloxy, etc., arylthio such as biphenylthio, etc., aralkyl such as biphenylmethyl, triphenylmethyl, etc.,

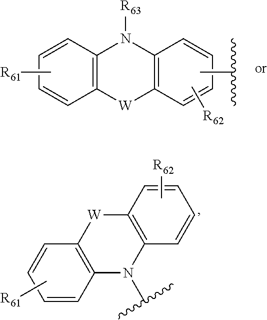

but are not limited thereto, and may be further substituted as shown in Chemical Formula 1.

More specifically, the R₁ through R₉ may be exemplified as following structures but are not limited thereto.

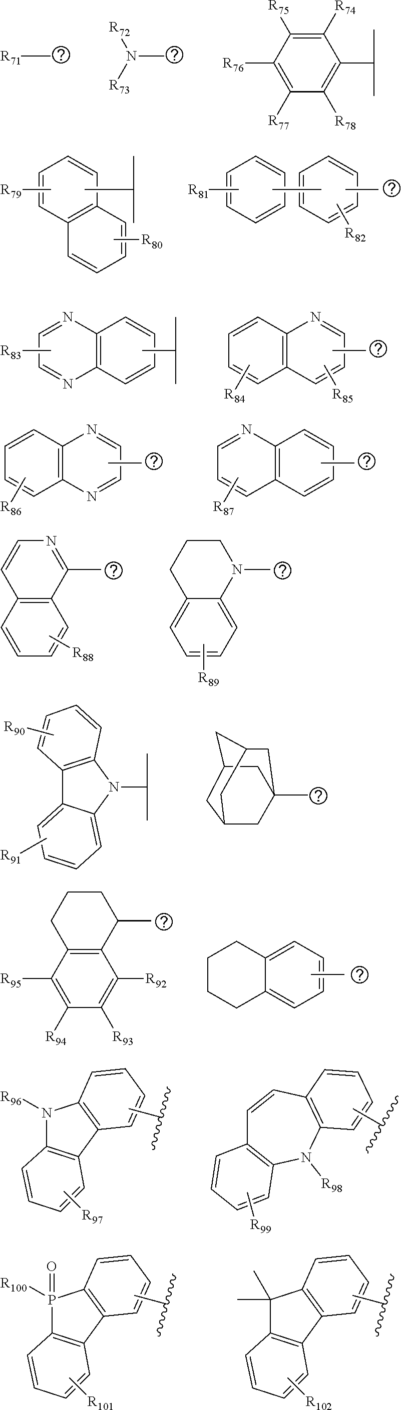

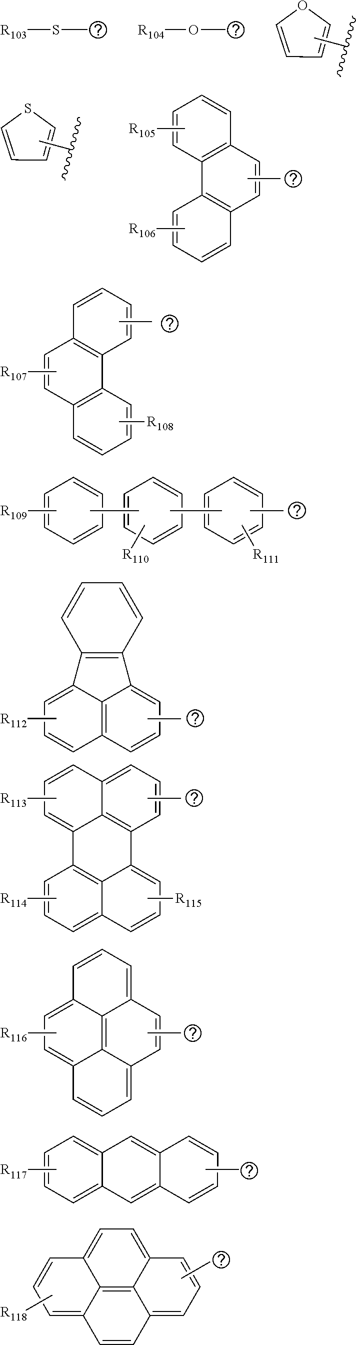

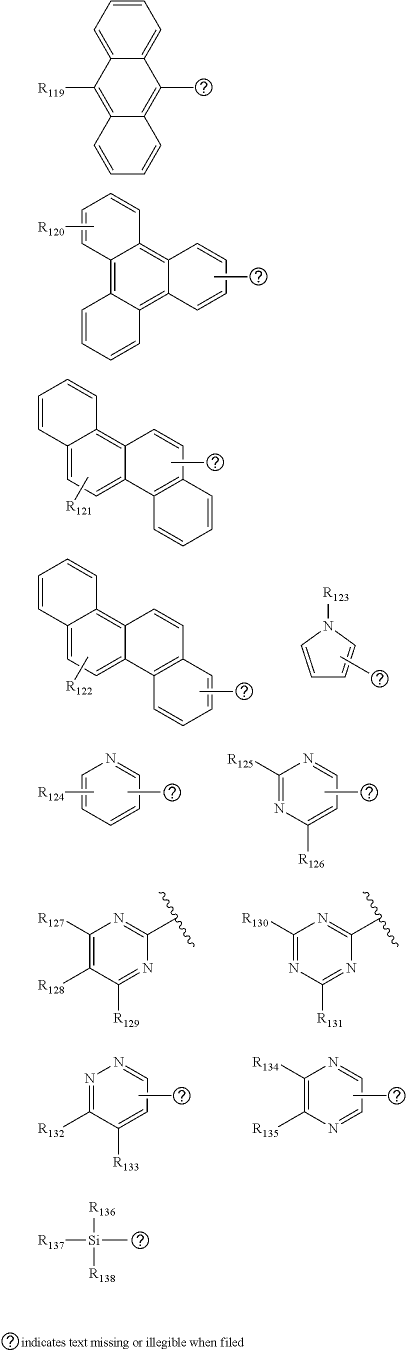

wherein

R₇₁ through R₁₃₈ independently represent hydrogen, deuterium, halogen, (C1-C30)alkyl, (C6-C30)aryl, (C6-C30)aryl fused with one or more (C3-C30)cycloalkyl(s), (C3-C30)heteroaryl, 5- to 7-membered heterocycloalkyl, 5- to 7-membered heterocycloalkyl fused with one or more aromatic ring(s), (C3-C30)cycloalkyl, (C3-C30)cycloalkyl fused with one or more aromatic ring(s), cyano, amino, (C1-C30)alkylamino, (C6-C30)arylamino, NR₄₁R₄₂, BR₄₃R₄₄, PR₄₅R₄₆, P(═O)R₄₇R₄₈, tri(C1-C30)alkylsilyl, di(C1-C30)alkyl(C6-C30)arylsilyl, tri(C6-C30)arylsilyl, (C6-C30)ar(C1-C30)alkyl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C2-C30)alkenyl, (C2-C30)alkynyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro or hydroxyl, or each of them may be linked to an adjacent substituent via (C3-C30)alkylene or (C3-C30)alkenylene with or without a fused ring to form an alicylic ring or a mono- or polycyclic aromatic ring, wherein R₄₁ through R₄₈ independently represent (C1-C30)alkyl, (C6-C30)aryl or (C3-C30)heteroaryl.

The

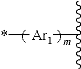

is exemplified as following structures but not limited thereto. m is the same as defined in Chemical Formula 1.

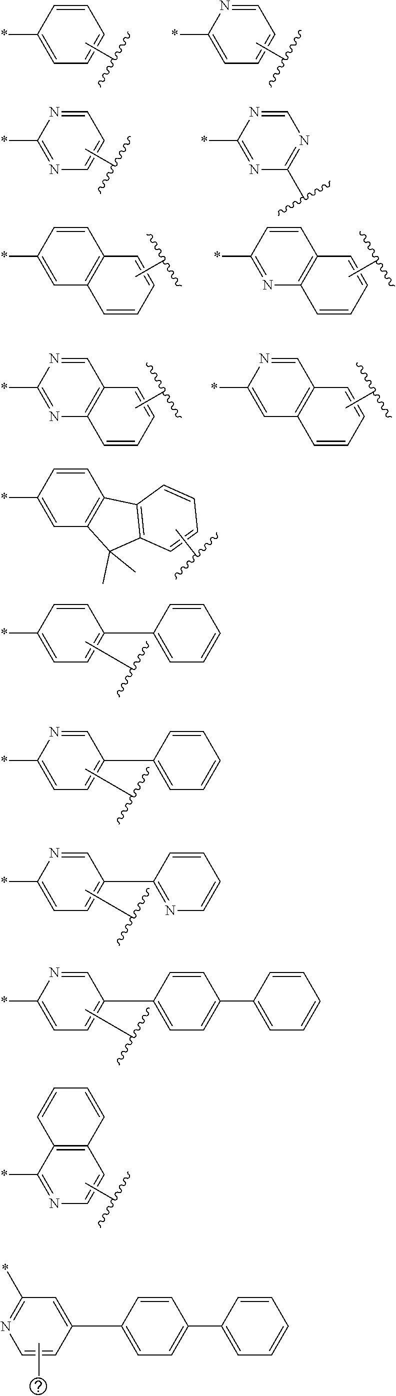

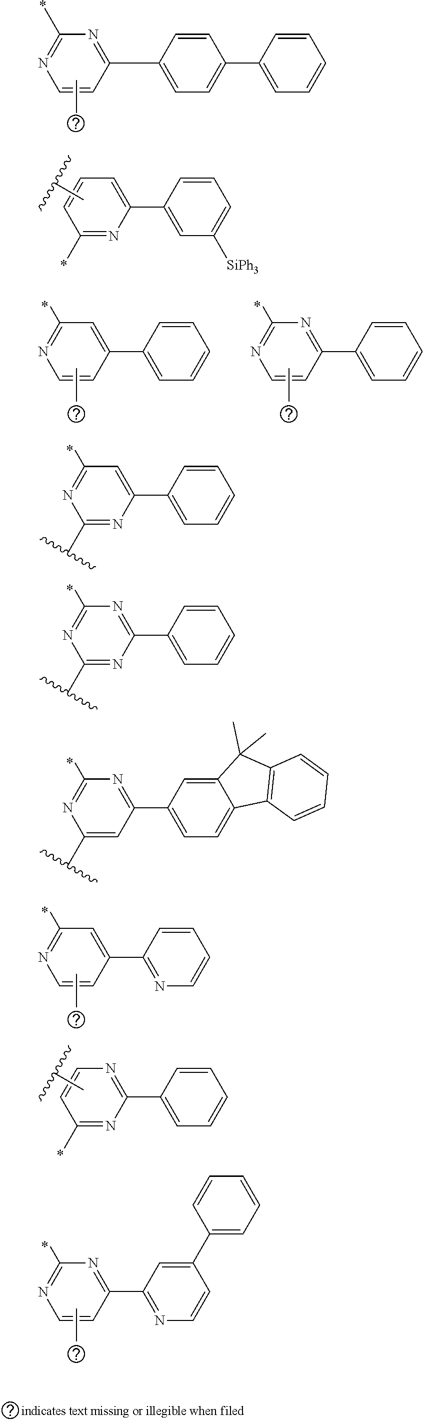

The organic electroluminescent compound according to the present invention may be specifically exemplified as following compounds but is not limited thereto.

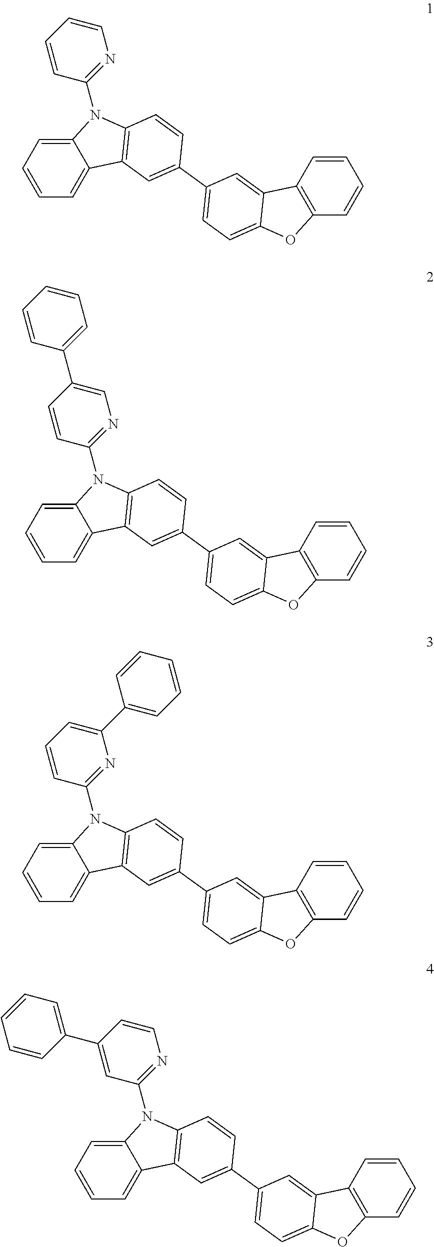

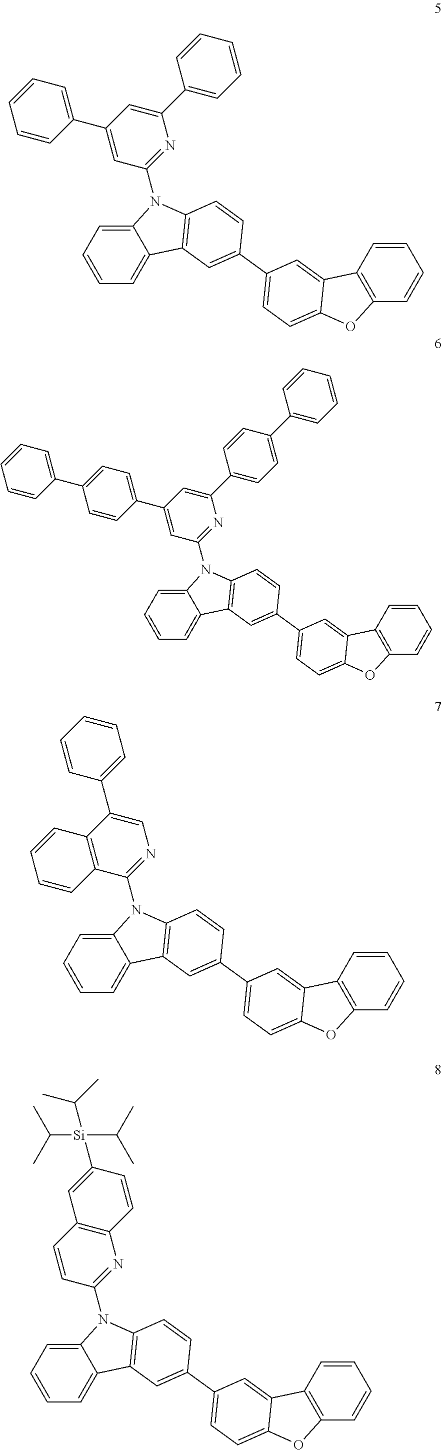

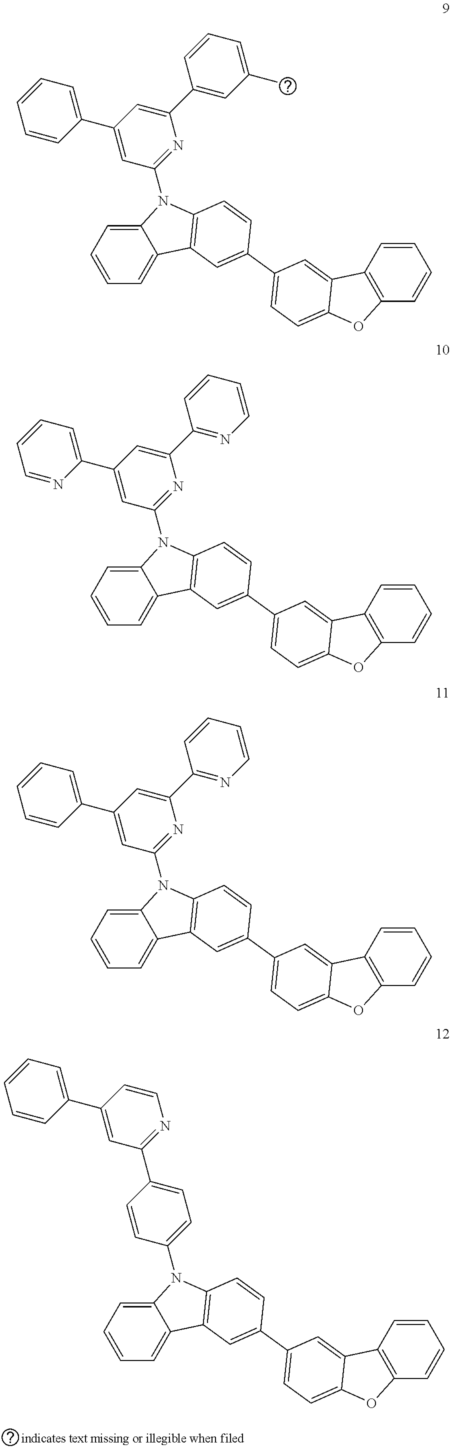

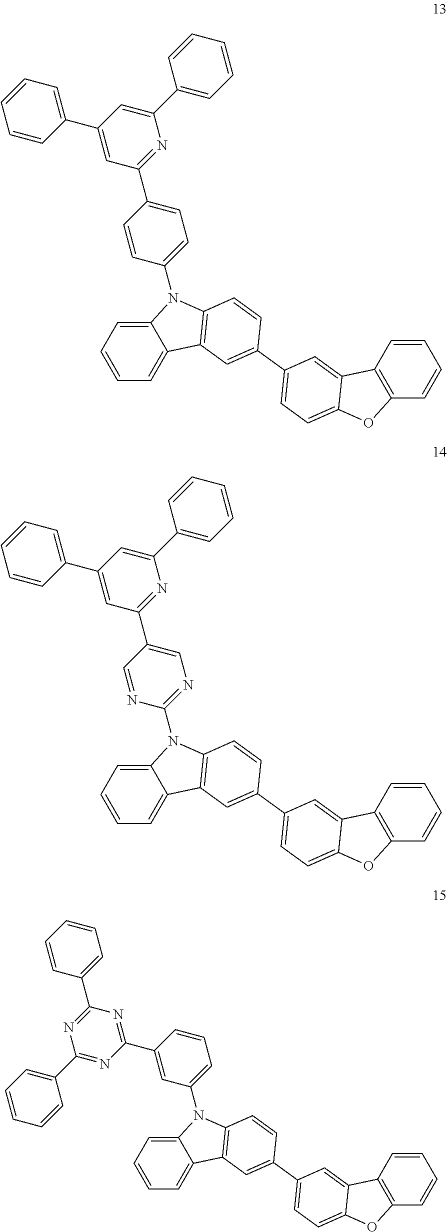

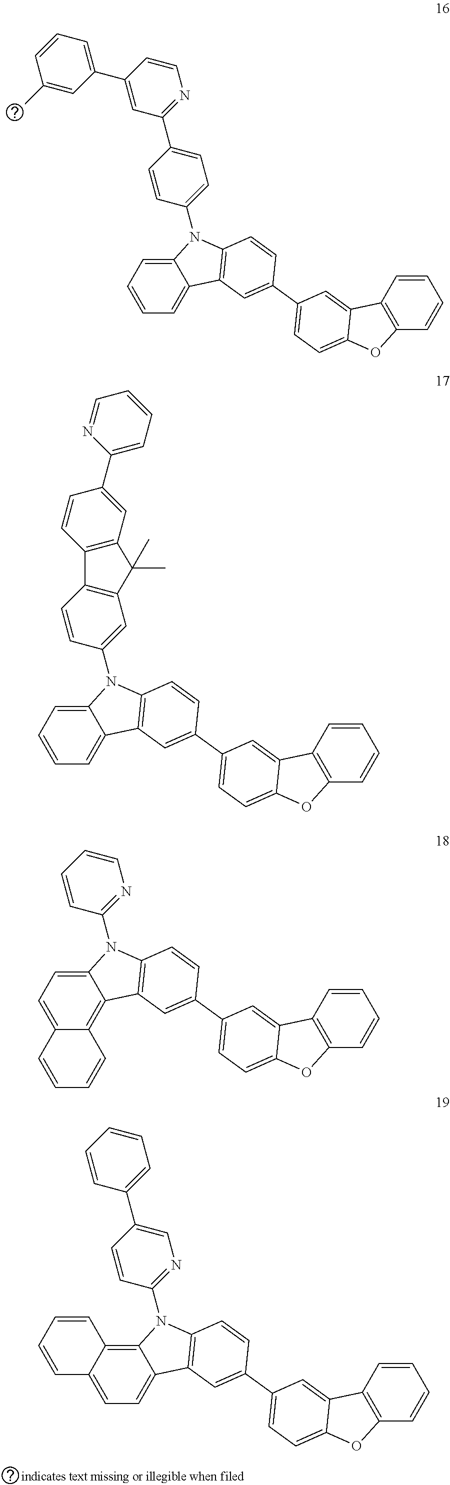

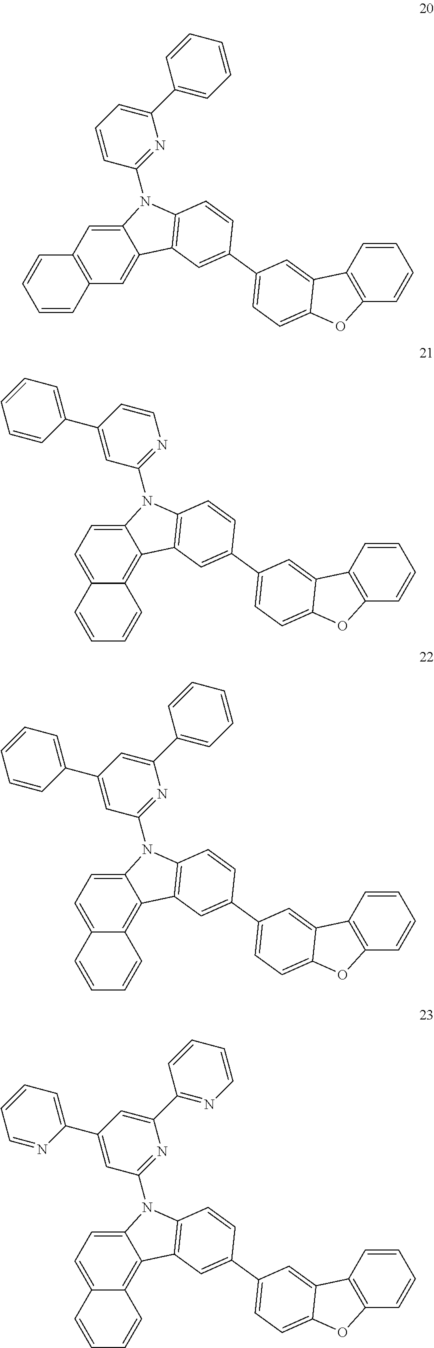

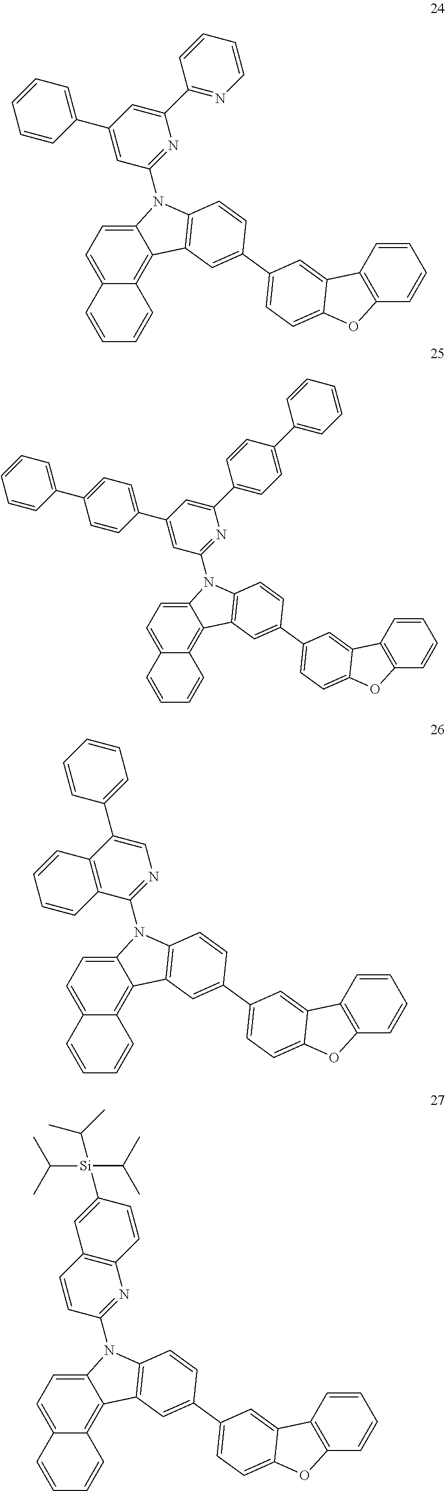

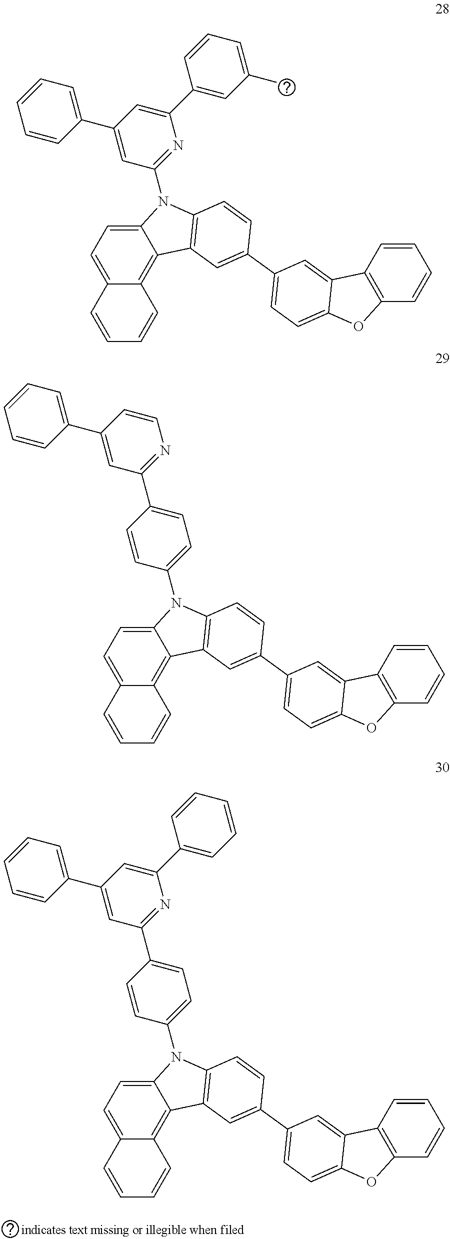

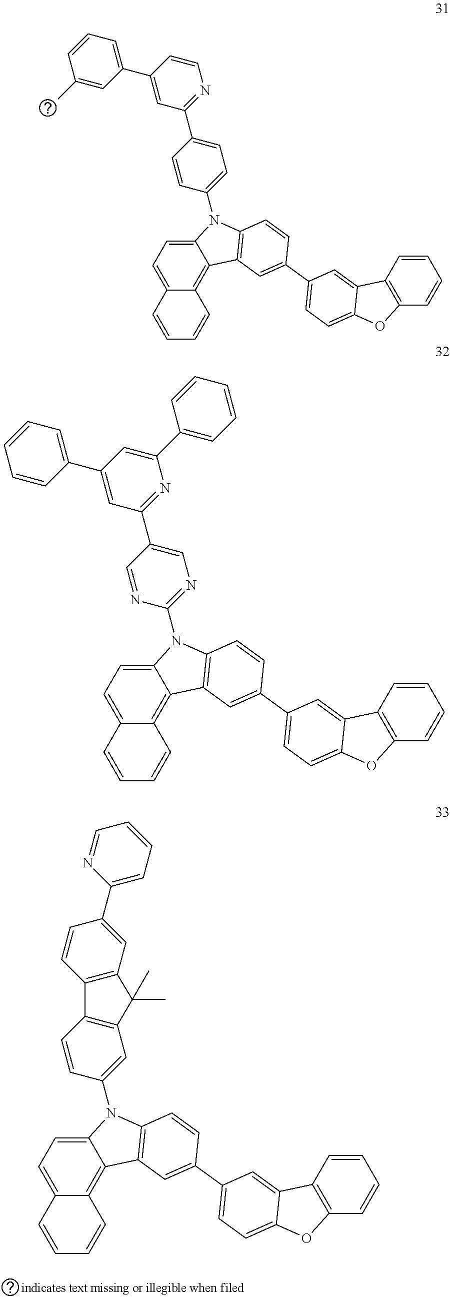

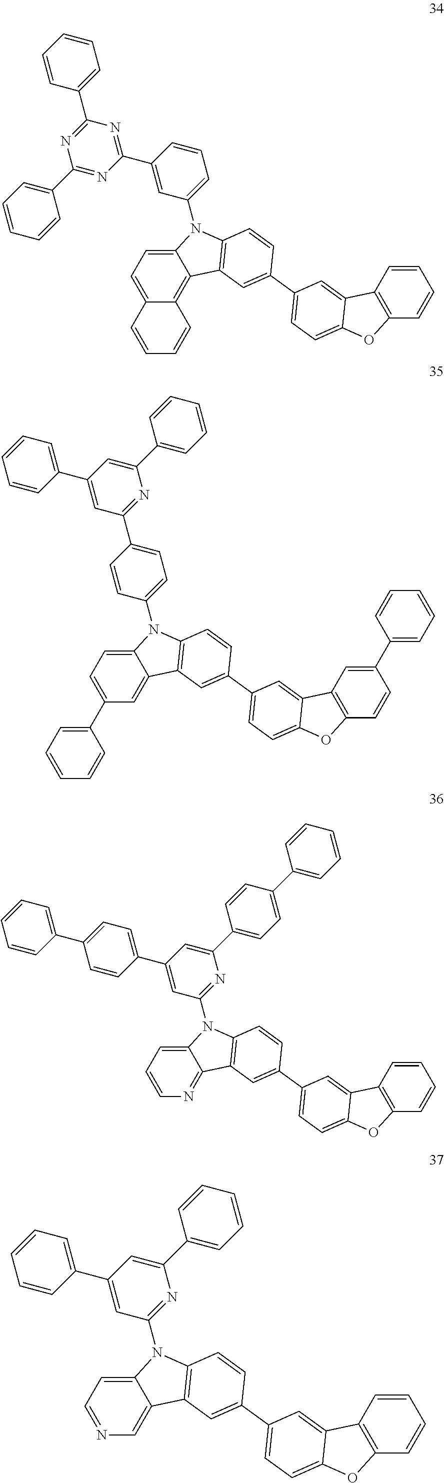

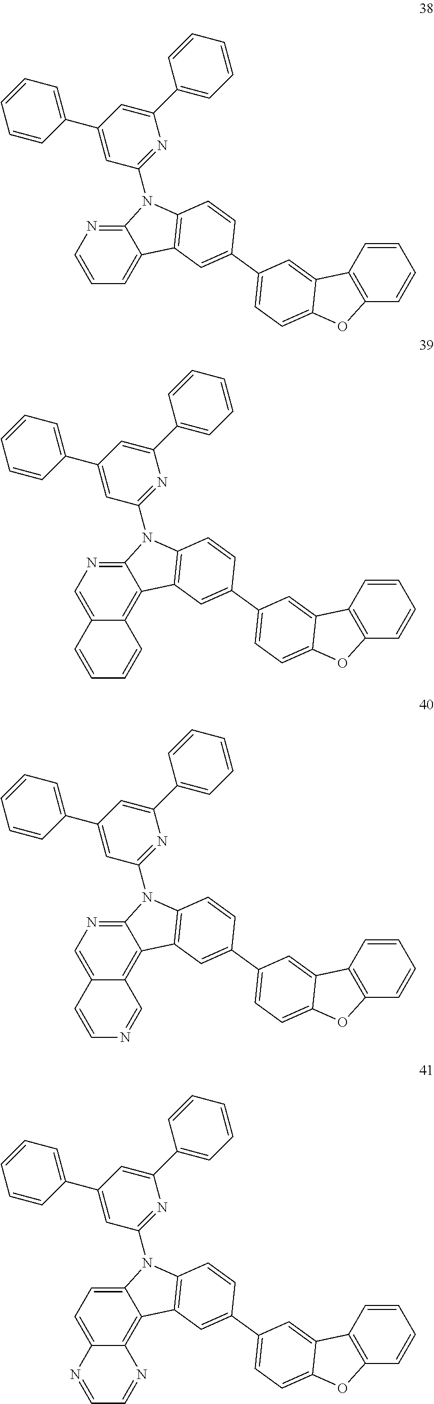

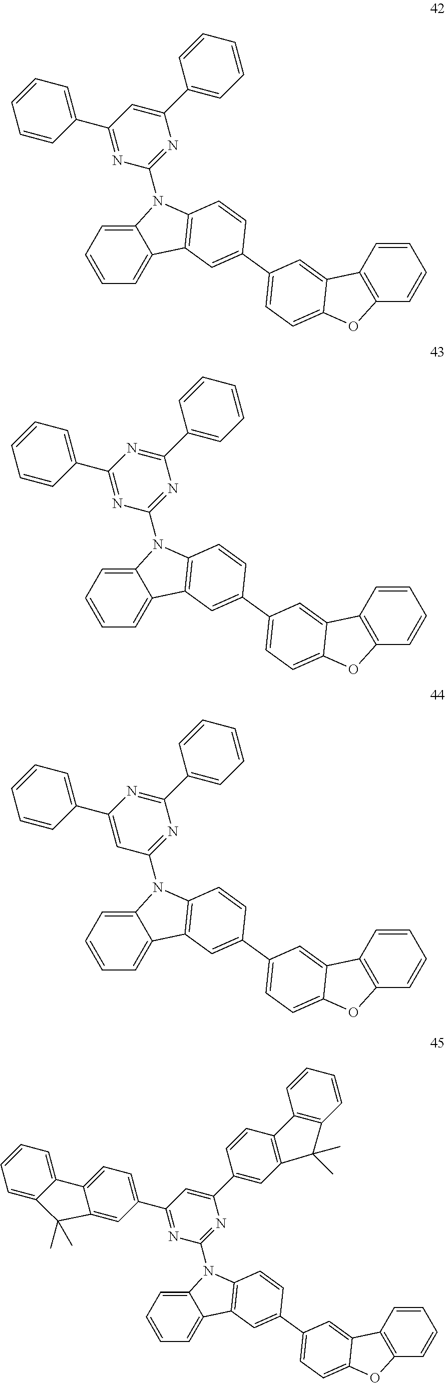

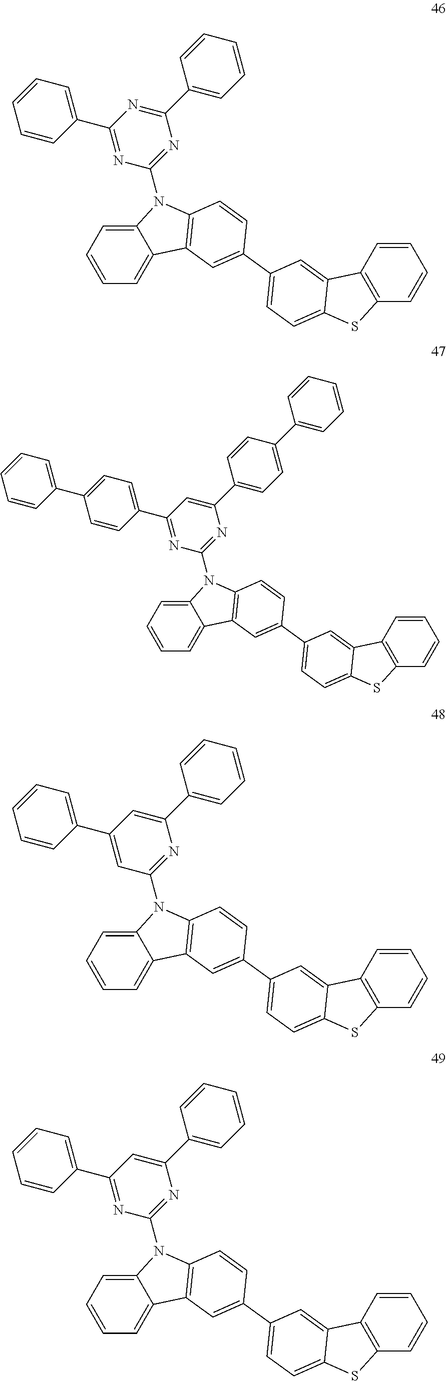

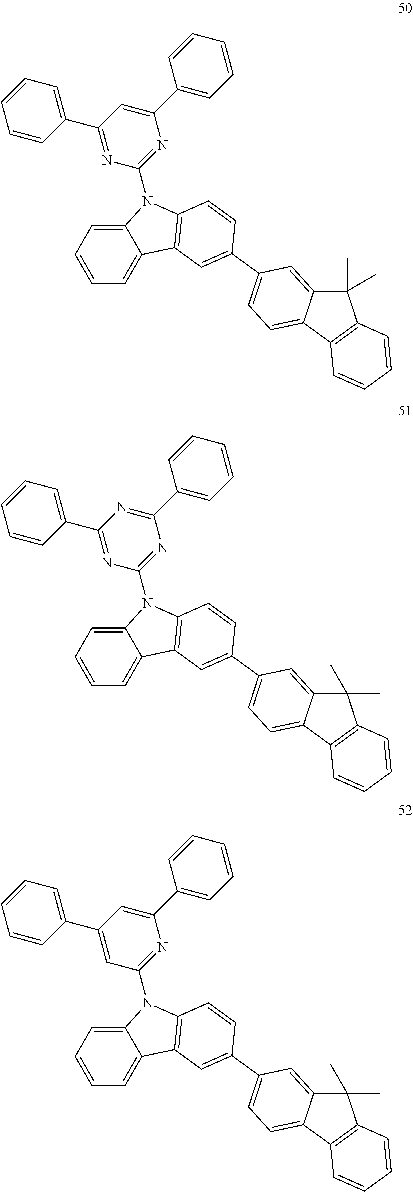

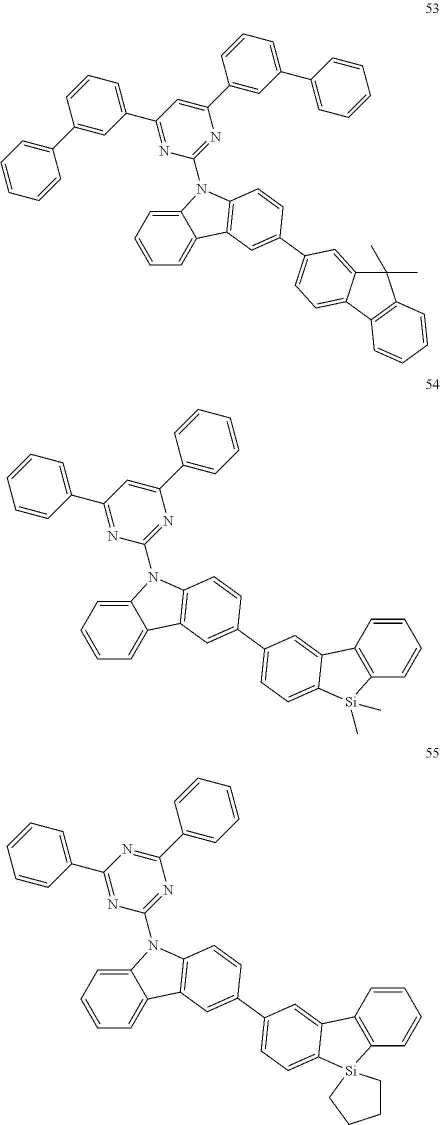

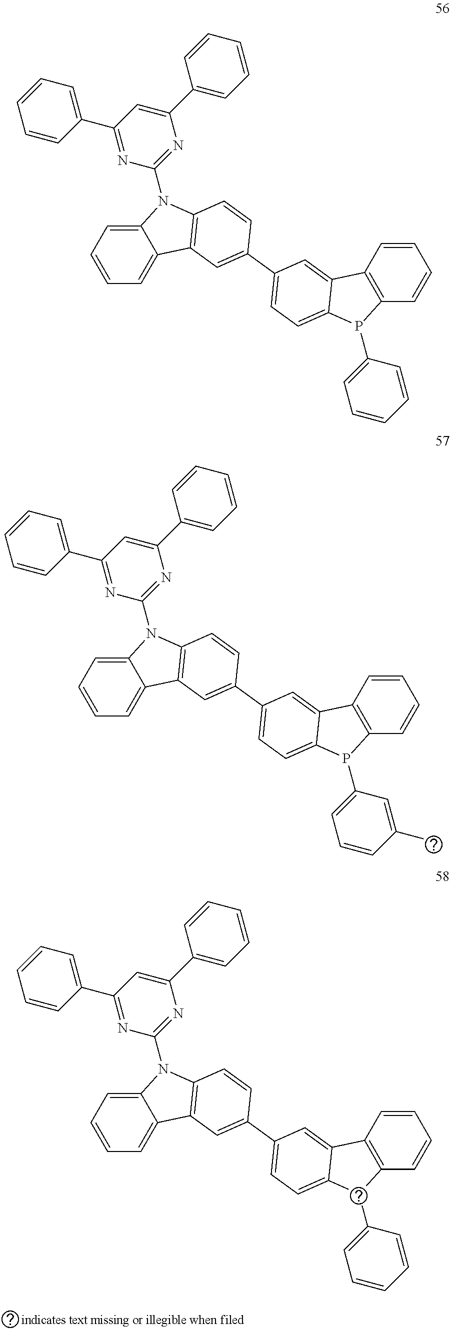

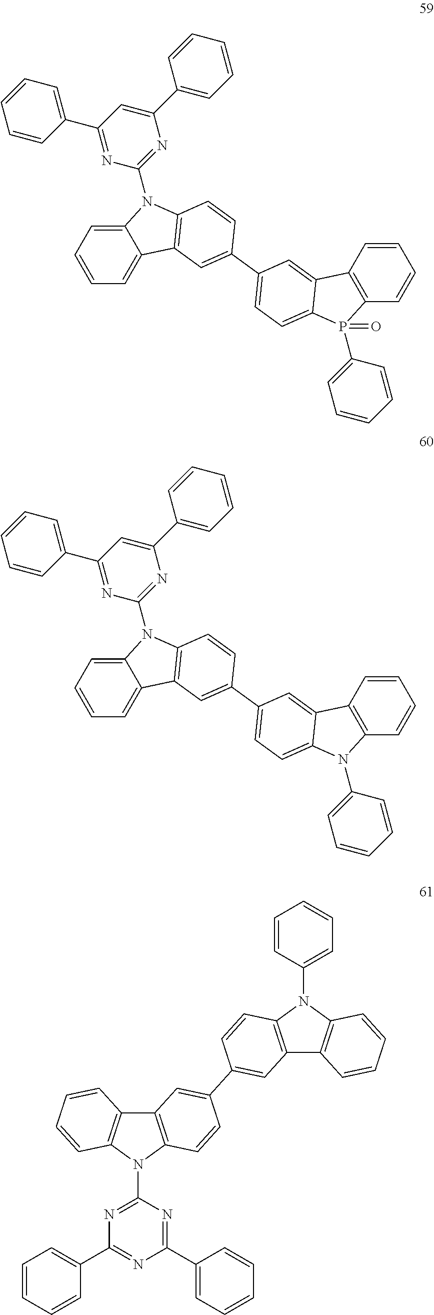

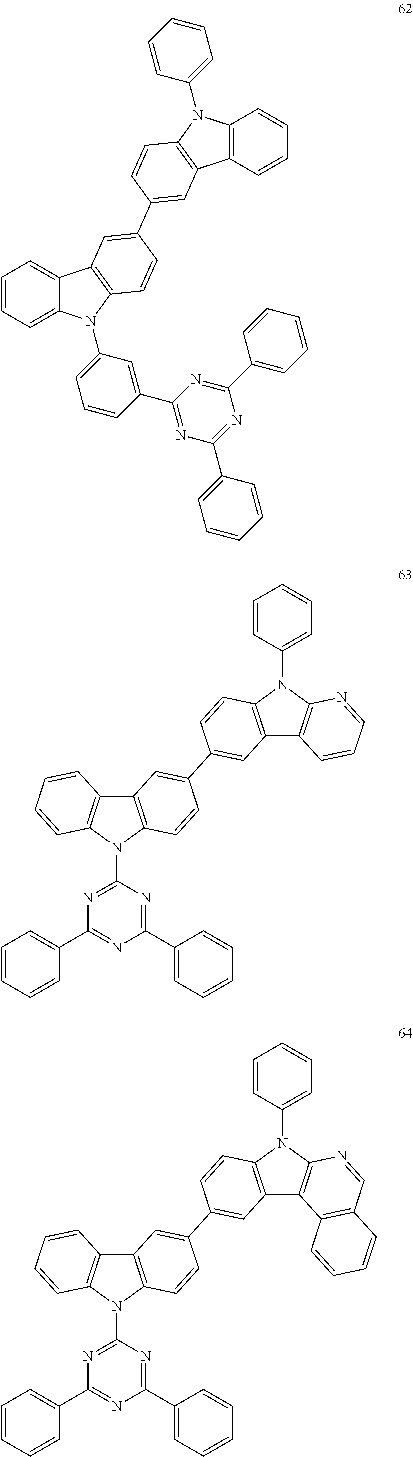

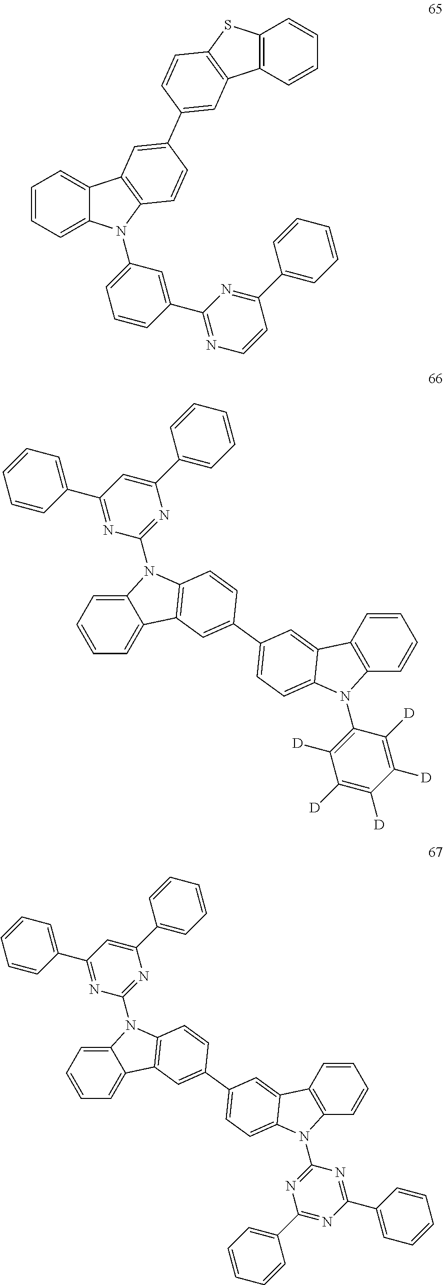

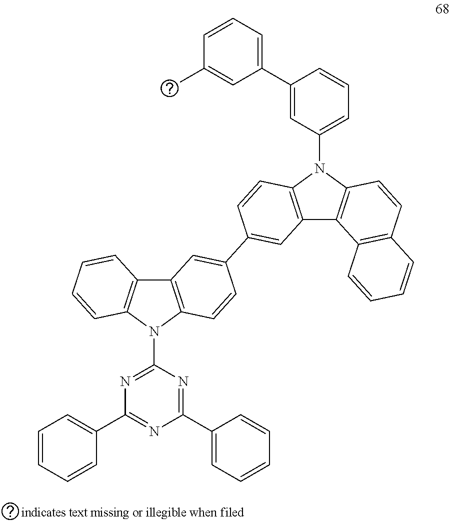

The organic electroluminescent compound according to the present invention may be prepared as shown in following Reaction Scheme 1.

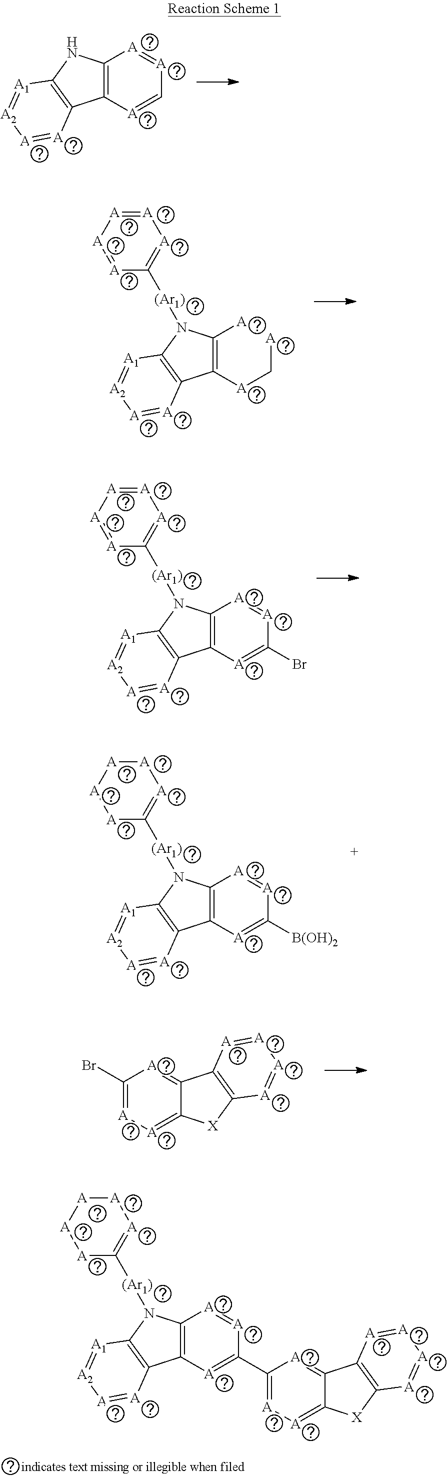

wherein

A₁ through A₁₉, X, Ar₁ and m are the same as defined in Chemical Formula 1.

Provided is an organic electroluminescent device, which comprises a first electrode; a second electrode; and one or more organic layer(s) interposed between the first electrode and the second electrode, wherein the organic layer comprises one or more organic electroluminescent compound(s) represented by Chemical Formula 1. The organic electroluminescent compound is used as a host material of the electroluminescent layer.

In addition, the organic layer may include the electroluminescent layer, and the electroluminescent layer may further include one or more dopants besides one or more organic electroluminescent compounds of Chemical Formula 1. The dopant applied to the organic electroluminescent device of the present invention is not specifically limited.

Preferably, the dopant applied to the organic electroluminescent device of the present invention is selected from following Chemical Formula 2.

M¹L¹⁰¹L¹⁰²L¹⁰³ Chemical Formula 2

wherein

M¹ is a metal selected from a group consisting of Group 7, Group 8, Group 9, Group 10, Group 11, Group 13, Group 14, Group 15 and Group 16 metals, and ligand L¹⁰¹, L¹⁰² and L¹⁰³ are independently selected from the following structures;

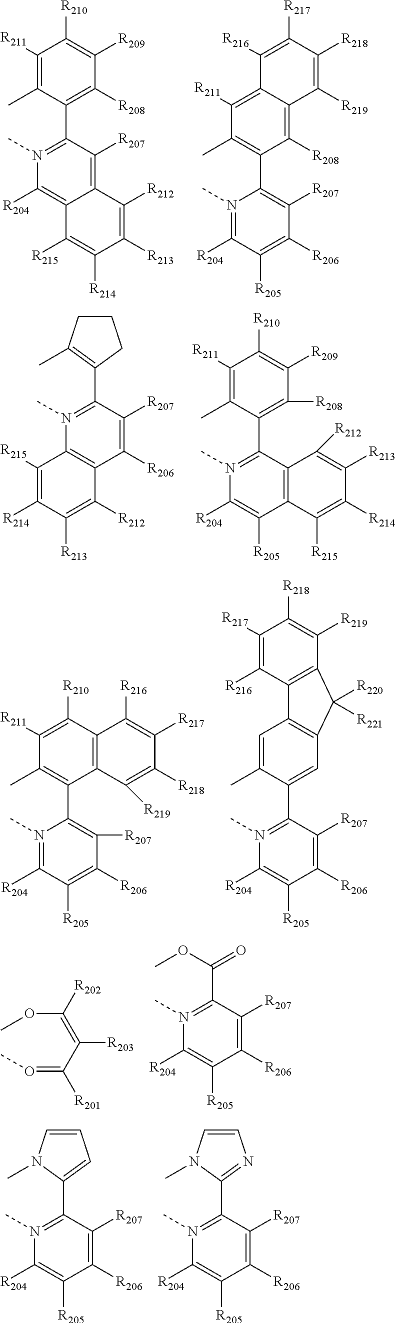

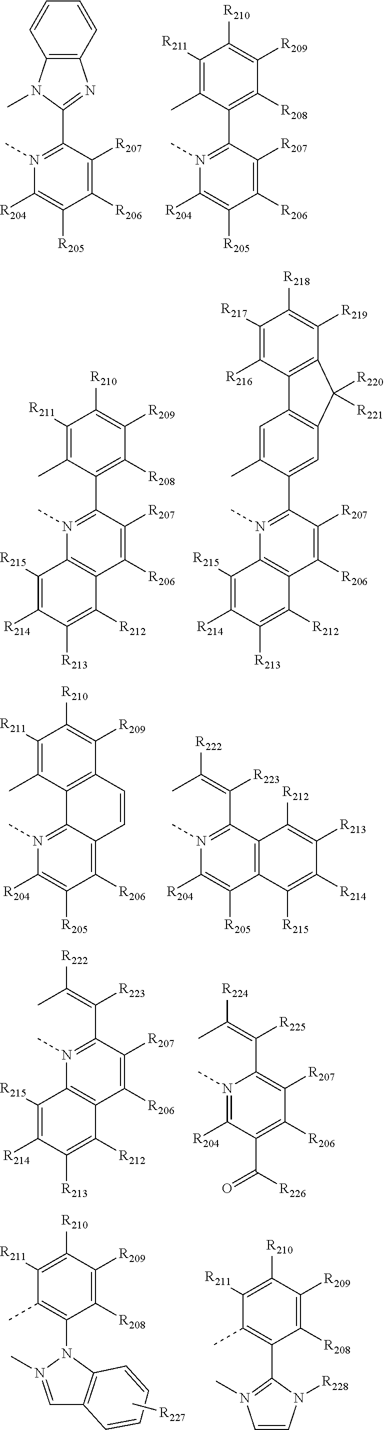

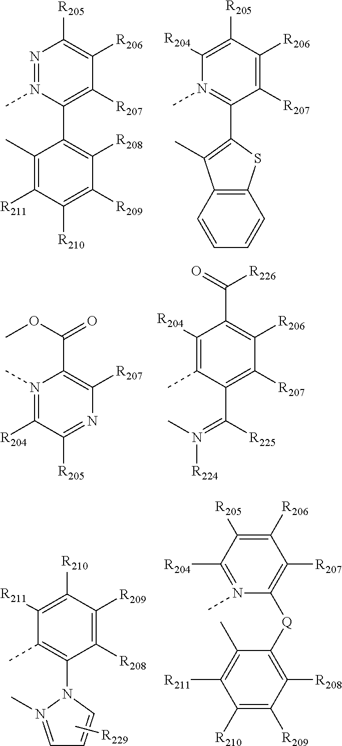

wherein

R₂₀₁ through R₂₀₃ independently represent hydrogen, (C1-C30)alkyl with or without halogen substituent(s), (C6-C30)aryl with or without (C1-C30)alkyl substituent(s) or halogen;

R₂₀₄ through R₂₁₉ independently represent hydrogen, (C1-C30)alkyl with or without substituent(s), (C1-C30)alkoxy with or without substituent(s), (C3-C30)cycloalkyl with or without substituent(s), (C2-C30)alkenyl with or without substituent(s), (C6-C30)aryl with or without substituent(s), mono- or di-(C1-C30)alkylamino with or without substituent(s), mono- or di-(C6-C30)arylamino with or without substituent(s), SF₅, tri(C1-C30)alkylsilyl with or without substituent(s), di(C1-C30)alkyl(C6-C30)arylsilyl with or without substituent(s), tri(C6-C30)arylsilyl with or without substituent(s), cyano or halogen;

R₂₂₀ through R₂₂₃ independently represent hydrogen, (C1-C30)alkyl with or without halogen substituent(s) or (C6-C30)aryl with or without (C1-C30)alkyl substituent(s);

R₂₂₄ and R₂₂₅ independently represent hydrogen, (C1-C30)alkyl with or without substituent(s), (C6-C30)aryl with or without substituent(s) or halogen, or R₂₂₄ and R₂₂₅ may be linked via (C3-C12)alkylene or (C3-C12)alkenylene with or without a fused ring to form an alicylic ring or a mono- or polycyclic aromatic ring;

R₂₂₆ represents (C1-C30)alkyl with or without substituent(s), (C6-C30)aryl with or without substituent(s), (C5-C30)heteroaryl with or without substituent(s) or halogen;

R₂₂₇ through R₂₂₉ independently represent hydrogen, (C1-C30)alkyl with or without substituent(s), (C6-C30)aryl with or without substituent(s) or halogen; and

Q represents

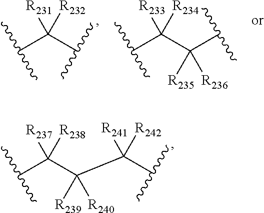

wherein R₂₃₁ through R₂₄₂ independently represent hydrogen, (C1-C30)alkyl with or without halogen substituent(s), (C1-C30)alkoxy, halogen, (C6-C30)aryl with or without substituent(s), cyano or (C5-C30)cycloalkyl with or without substituent(s), or each of them may be linked to an adjacent substituent via alkylene or alkenylene to form a spiro ring or a fused ring, or may be linked to R₂₀₇ or R₂₀₈ via alkylene or alkenylene to form a saturated or unsaturated fused ring.

The meaning of the electroluminescent layer may be a single layer as a layer where the light is emitted or may be a multiple layer where two or more layers are laminated. In the configuration of the present invention, when host-dopant are used in mixture, it is confirmed that the luminous efficiency are remarkably improved by the electroluminescent host of the present invention. It may be configured at doping concentration of 0.5 to 10 wt %. Compared to other host materials, the electroluminescent host of the present invention has superior conductivity with respect to the hole and electron and excellent stability in material, thereby showing a characteristic of remarkably increasing its life span as well as improving the luminous efficiency.

The M¹ is selected from a group consisting of Ir, Pt, Pd, Rh, Re, Os, Tl, Pb, Bi, In, Sn, Sb, Te, Au and Ag. The dopant compounds of Chemical Formula 2 are exemplified by the compounds described in Korean Patent Application No. 10-2008-0112855, but are not limited thereto.

In the organic electronic device of the present invention, the organic layer may further include, in addition to the organic electroluminescent compound represented by Chemical Formula 1, one or more compound(s) selected from a group consisting of arylamine compounds and styrylarylamine compounds, at the same time. The arylamine compounds or styrylarylamine compounds are exemplified in Korean Patent Application No. 10-2008-0123276, 10-2008-0107606 or 10-2008-0118428, but are not limited thereto.

Further, in the organic electroluminescent device of the present invention, the organic layer may further include, in addition to the organic electroluminescent compound represented by Chemical Formula 1, one or more metal(s) selected from a group consisting of organic metals of Group 1, Group 2, 4th period and 5th period transition metals, lanthanide metals and d-transition elements or complex compound(s). The organic layer may include an electroluminescent layer and a charge generating layer.

Further, the organic layer may include, in addition to the organic electroluminescent compound of Chemical Formula 1, one or more organic electroluminescent layer(s) emitting blue, green or red light at the same time in order to embody a white-emitting organic electroluminescent device. The compound emitting blue, green or red light may be exemplified by the compounds described in Korean Patent Application No. 10-2008-0123276, 10-2008-0107606 or 10-2008-0118428, but are not limited thereto.

In the organic electroluminescent device of the present invention, a layer (hereinafter referred to as “surface layer”) selected from a chalcogenide layer, a metal halide layer and a metal oxide layer may be placed on the inner surface of one or both electrode(s) among the pair of electrodes. More specifically, a metal chalcogenide (including oxide) layer of silicon or aluminum may be placed on the anode surface of the electroluminescent medium layer, and a metal halide layer or metal oxide layer may be placed on the cathode surface of the electroluminescent medium layer. Operation stability may be attained therefrom.

The chalcogenide may be, for example, SiOₓ (1≦x≦2), AlOₓ (1≦x≦1.5), SiON, SiAlON, etc. The metal halide may be, for example, LiF, MgF₂, CaF₂, a rare earth metal fluoride, etc. The metal oxide may be, for example, Cs₂O, Li₂O, MgO, SrO, BaO, CaO, etc.

In the organic electroluminescent device according to the present invention, it is also preferable to arrange on at least one surface of the pair of electrodes thus manufactured a mixed region of an electron transport compound and a reductive dopant, or a mixed region of a hole transport compound and an oxidative dopant. In that case, since the electron transport compound is reduced to an anion, injection and transport of electrons from the mixed region to an electroluminescent medium are facilitated. In addition, since the hole transport compound is oxidized to a cation, injection and transport of holes from the mixed region to an electroluminescent medium are facilitated. Preferable oxidative dopants include various Lewis acids and acceptor compounds. Preferable reductive dopants include alkali metals, alkali metal compounds, alkaline earth metals, rare-earth metals, and mixtures thereof.

Further, a white-emitting electroluminescent device having two or more electroluminescent layers may be manufactured by employing a reductive dopant layer as a charge generating layer.

### ADVANTAGEOUS EFFECTS

Since the organic electroluminescent compound according to the present invention exhibits good luminous efficiency and excellent life property, it may be used to manufacture OLED devices having very superior operation life.

### MODE FOR INVENTION

The present invention is further described with respect to organic electroluminescent compounds according to the present invention, processes for preparing the same, and luminescence properties of devices employing the same. However, the following examples are provided for illustrative purposes only and they are not intended to limit the scope of the present invention.

### Preparation Example 1

### Preparation of Compound 1

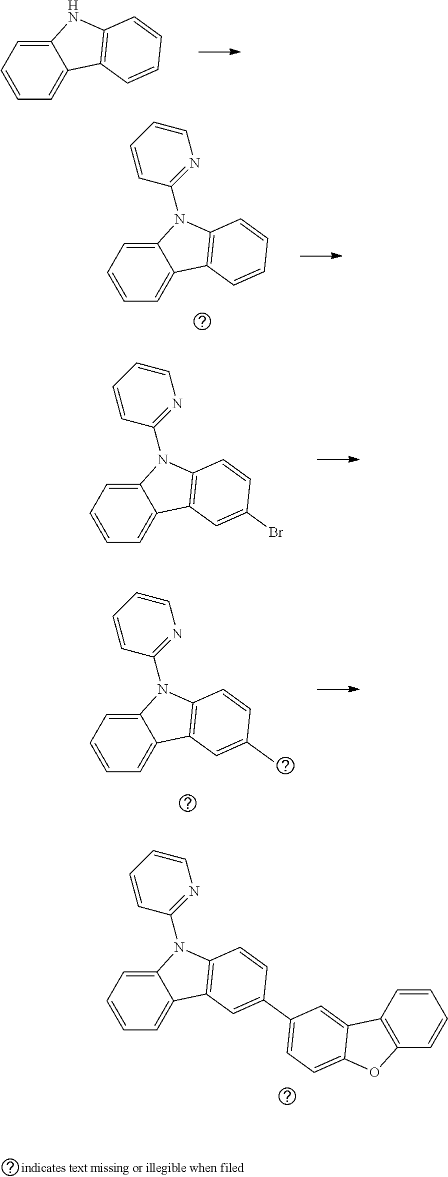

Preparation of Compound 1-1

9H-carbazole (10 g, 41.10 mmol), 2-chloropyridine (5.60 g, 49.32 mmol), Pd(OAc)₂ (0.46 g), NaOt-bu (7.9 g, 82.20 mmol), toluene (100 mL), P(t-bu)₃ (2 mL, 4.11 mmol, 50% in toluene) were added and stirred under reflux. 10 hours later, the mixture was cooled to room temperature and distilled water was added. Extracting with EA and drying with MgSO₄, drying under reduce pressure was performed. Compound 1-1 (8.3 g, 33.98 mmol, 83%) was obtained via column separation.

Preparation of Compound 1-2

1-neck flask was filled with Compound 1-1 (8.3 g, 33.98 mmol), formed a vacuum and was filled with argon. After tetrahydrofuran (500 mL) was added, the mixture was stirred at 0° C. for 10 minutes. NBS (7.35 g, 40.78 mmol) was added thereto and stirred at room temperature for one day. Upon completion of the reaction, the product was extracted with distilled water and EA. After drying an organic layer with MgSO₄ and removing solvent by a rotary type evaporator, Compound 1-2 (8.5 g, 26.30 mmol, 77%) was obtained via column chromatography using hexan and EA as developing solvent.

Preparation of Compound 1-3

The 1-neck flask was filled with Compound 1-2 (8.5 g, 26.30 mmol), formed a vacuum and was filled with argon. After tetrahydrofuran (500 mL) was added, the mixture was stirred at 78° C. for 10 minutes. n-BuLi(2.5M in hexane) (15.8 mL, 39.45 mmol) was added dropwise and stirred at −78° C. for 1 hour and a half. Trimethylborate (4.85 mL, 39.45 mmol) was added at −78° C. The mixture was stirred at −78° C. for 30 minutes and stirred at room temperature for 4 hours. Upon completion of the reaction, the product was extracted with distilled water and EA. After drying an organic layer with MgSO₄ and removing solvent by a rotary type evaporator, Compound 1-3 (5.2 g, 18.05 mmol, 68.6%) was obtained via column chromatography using hexan and EA as developing solvent.

Preparation of Compound 1

Compound 1-3 (5.0 g, 17.4 mmol), 2-bromodibenzo[b,d]furan (5.2 g, 20.88 mmol), Pd(PPh₃)₄ (0.8 g, 0.7 mmol), 2M K₂CO₃ aqueous solution (20 mL), toluene (100 mL), and ethanol (50 mL) were added and stirred under reflux for 12 hours. After washing with distilled water, extracting with EA, and drying with MgSO₄, distillation under reduced pressure followed by column separation yielded Compound 1 (4.3 g, 10.48 mmol, 60%).

### Preparation Example 2

### Preparation of Compound 49

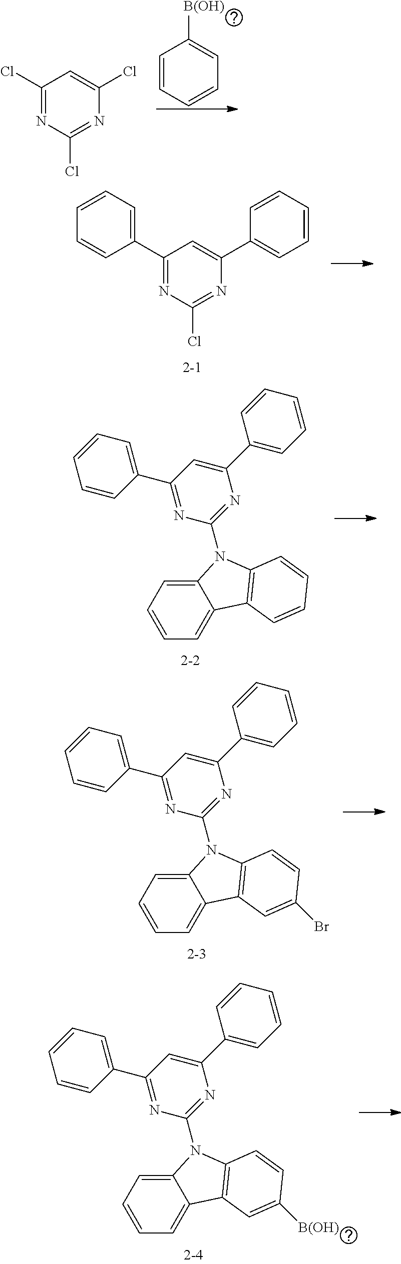

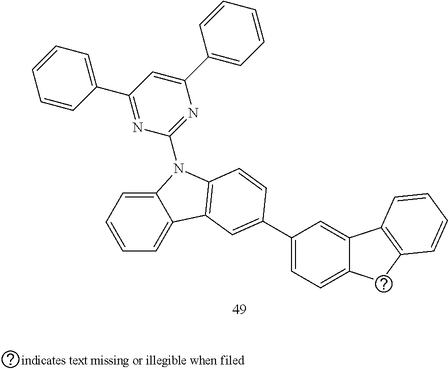

Preparation of Compound 2-1

2,4,6-trichloropyrimidine (10 g, 54.51 mmol), phenylboronic acid (16.6 g, 136.29 mmol), Pd(PPh₃)₄ (3.15 g, 2.72 mmol), 2M K₂CO₃ (50 mL), toluene (100 mL), and ethanol (30 mL) were added and stirred under reflux. 4 hours later, the mixture was cooled to room temperature and distilled water was added thereto. After extracting with EA and drying with MgSO₄, distillation under reduced pressure followed by column separation yielded Compound 2-1 (7 g, 26.24 mmol, 48.14%).

Preparation of Compound 2-2

NaH (1.57 g, 39.36 mmol, 60% in mineral oil) was mixed with DMF (70 mL) and Compound 2-1 (7 g, 26.24 mmol) was dissolved in DMF (60 mL). 1 hour later, Compound 9H-carbazole was dissolved in DMF (70 mL). The mixture was stirred for 10 hours. After adding distilled water, extracting with EA, and drying with MgSO₄, distillation under reduced pressure followed by column separation yielded Compound 2-2 (7 g, 14.78 mmol, 56.33%).

Preparation of Compound 2-3

Compound 2-3 (5.7 g, 11.97 mmol, 80.9%) was obtained by combining Compound 2-2 (7 g, 14.78 mmol) in Preparation Example 1 according to the same method as the preparation of Compound 1-2.

Preparation of Compound 2-4

Compound 2-4 (3.4 g, 7.70 mmol, 64.4%) was obtained by combining Compound 2-3 (5.7 g, 11.97 mmol) in Preparation Example 1 according to the same method as the preparation of Compound 1-3.

Preparation of Compound 49

Compound 49 (3.2 g, 5.52 mmol, 72%) was obtained by using Compound 2-4 (3.4 g, 7.70 mmol) and 2-bromodibenzo[b,d]thiophene in Preparation Example 1 according to the same method as the preparation of Compound 1.

### Preparation Example 3

### Preparation of Compound 51

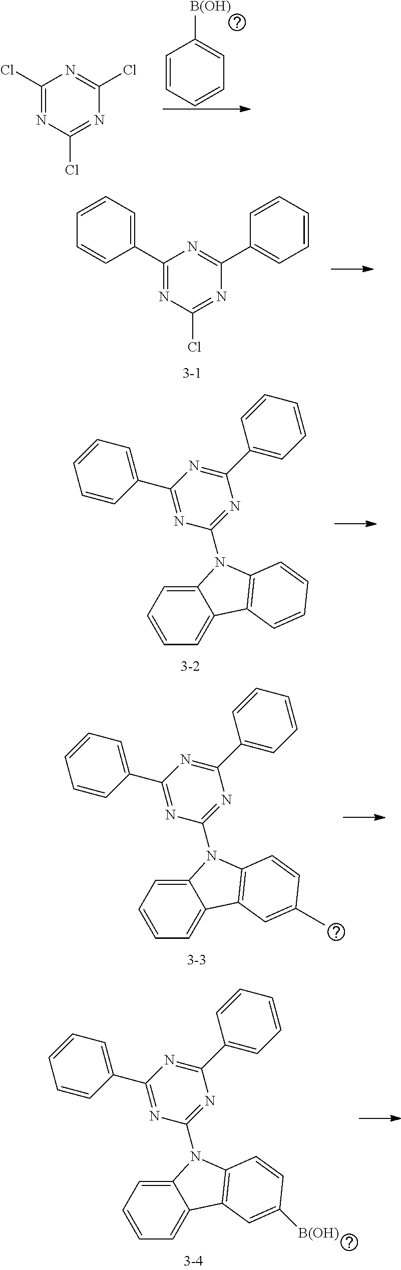

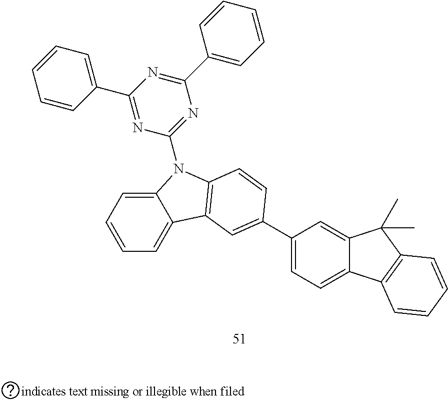

Preparation of Compound 3-1

Compound 3-1 (13.2 g, 47.7 mmol, 87.5%) was obtained by combining 2,4,6-trichlorotriazine (10 g, 54.51 mmol) in Preparation Example 2 according to the same method as the preparation of Compound 2-1.

Preparation of Compound 3-2

Compound 3-2 (14.5 g, 36.39 mmol, 76.3%) was obtained by combining Compound 3-1 (13.2 g, 47.7 mmol) in Preparation Example 2 according to the same method as the preparation of Compound 2-2.

Preparation of Compound 3-3

Compound 3-3 (14.6 g, 30.59 mmol, 84%) was obtained by combining Compound 3-2 (14.5 g, 36.39 mmol) in Preparation Example 2 according to the same method as the preparation of Compound 2-3.

Preparation of Compound 3-4

Compound 3-4 (7.2 g, 16.28 mmol, 53.2%) was obtained by combining Compound 3-3 (14.6 g, 30.59 mmol) in Preparation Example 2 according to the same method as the preparation of Compound 2-4.

Preparation of Compound 51

Compound 51 (5.1 g, 8.63 mmol, 53%) was obtained by using Compound 3-4 (7.2 g, 16.28 mmol) and 2-bromo-9,9-dimethyl-9H-fluorene in Preparation Example 2 according to the same method as the preparation of Compound 49.

### Preparation Example 4

### Preparation of Compound 62

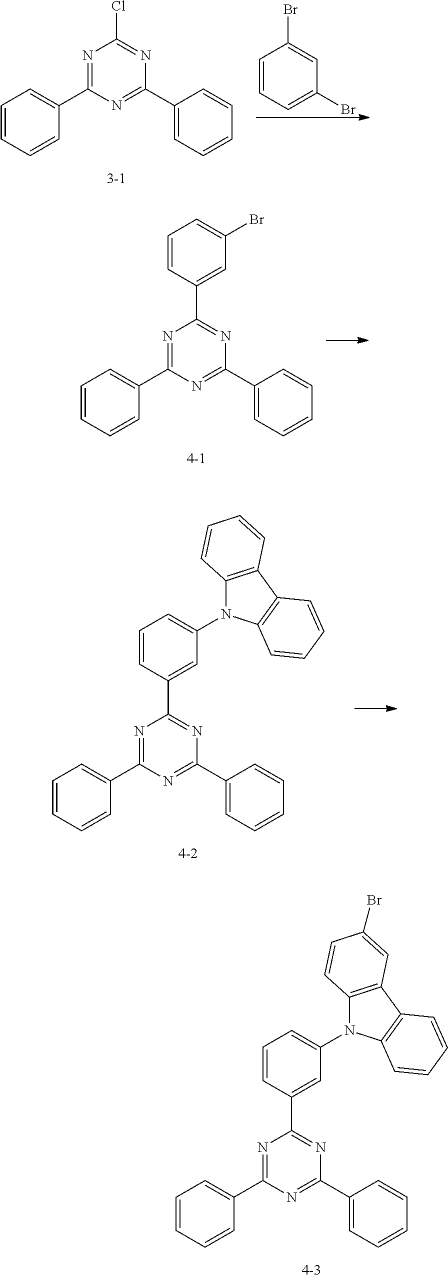

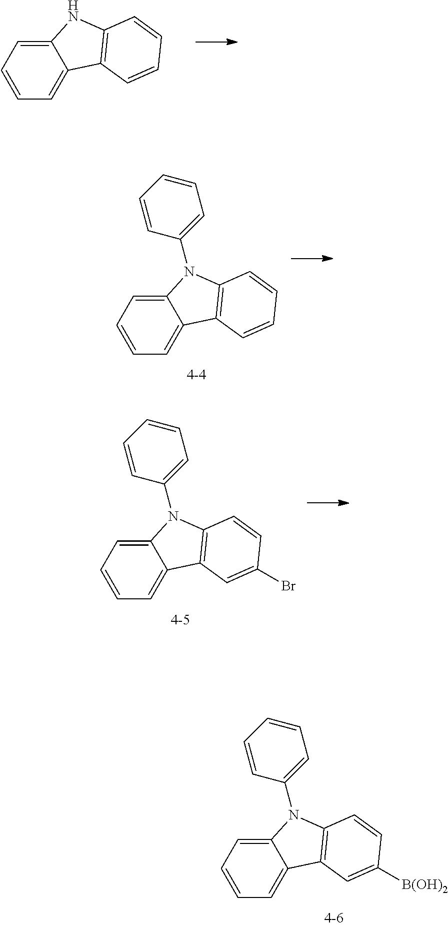

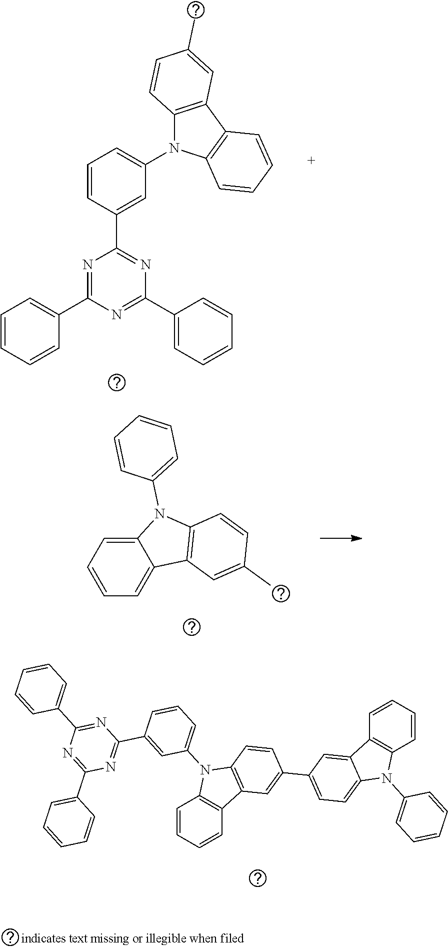

Preparation of Compound 4-1

1,3-dibromobenzene (28 g, 0.119 mol) was dissolved in THF (600 mL) and n-BuLi (47.5 mL) was slowly added dropwise at −78° C. After reacting and stirring for 1 hour, Compound 3-1 (47.5 mL) was slowly added dropwise and slowly heated. The mixture was stirred at room temperature for 5 hours. Upon completion of the reaction, the product was extracted with EA and distilled water. Compound 4-1 (15.7 g, 40.43 mmol, 40.4%) was obtained via column separation.

Preparation of Compound 4-2

Compound 4-2 (12.5 g, 26.34 mmol, 65.2%) was obtained by combining Compound 4-1 (15.7 g, 40.43 mmol) in Preparation Example 1 according to the same method as the preparation of Compound 1-1.

Preparation of Compound 4-3

Compound 4-3 (9.8 g, 17.71 mmol, 67.3%) was obtained by combining Compound 4-2 (12.5 g, 26.34 mmol) in Preparation Example 1 according to the same method as the preparation of Compound 1-2.

Preparation of Compound 4-4

9H-carbazole (70 g, 0.42 mmol), Iodobenzene (46 mL), cooper (40 g), potassiumcarbonate (174 g), 18-crown-6 (9 g), and 1,2-Dichlorobenzene (2 L) were added and stirred under reflux for 12 hours. Upon completion of the reaction, the product was extracted with EA and dried with MgSO₄. Distillation under reduced pressure followed by column separation yielded Compound 4-4 (63.4 g, 260.58 mmol, 62%).

Preparation of Compound 4-5

Compound 4-5 (52.4 g, 162.63 mmol, 62.4%) was obtained by combining Compound 4-4 (63.4 g, 260.58 mmol) in Preparation Example 1 according to the same method as the preparation of Compound 1-2.

Preparation of Compound 4-6

Compound 4-6 (20.3 g, 70.70 mmol, 43%) was obtained by combining Compound 4-5 (52.4 g, 162.63 mmol) in Preparation Example 1 according to the same method as the preparation of Compound 1-3.

Preparation of Compound 62

Compound 62 (5.7 g, 7.96 mmol, 50%) was obtained by using Compound 4-3 (9.8 g, 17.71 mmol) and Compound 4-6 in Preparation Example 1 according to the same method as the preparation of Compound 1.

Organic electroluminescent Compounds 1 to 68 were prepared according to Preparation Examples 1 to 4 and Table 1 shows ¹H NMR and MS/FAB of the prepared organic electroluminescent compounds.

TABLE 1

| | | MS/FAB | |
|---|---|---|---|
| Cmpd. | ¹H NMR (CDCl₃, 200 MHz) | found | calculated |
| 1 | δ = 7.25 (1H, m), 7.32~7.4 (4H, m), | 410.47 | 410.14 |
| | 7.66~7.81 (6H, m), 7.87~7.94 (4H, m), 8.01 (1H, | | |
| | m), 8.41 (1H, m), 8.55 (1H, m) | | |
| 4 | δ = 7.25 (1H, m), 7.32~7.41 (4H, m), | 486.56 | 486.17 |
| | 7.51~7.52 (5H, m), 7.66~7.81 (6H, m), | | |
| | 7.87~7.94 (3H, m), 8.4 (1H, m), 8.47 (1H, m), | | |
| | 8.55 (1H, m) | | |
| 5 | δ = 7.11 (1H, m), 7.25 (1H, m), 7.32~7.41 (11H, | 562.66 | 562.20 |
| | m), 7.66~7.81 (6H, m), 7.87~7.94 (3H, m), | | |
| | 8.3 (2H, m), 8.55~8.6 (2H, m) | | |
| 7 | δ = 7.25 (1H, m), 7.32~7.42 (5H, m), | 536.62 | 536.19 |
| | 7.49~7.52 (5H, m), 7.66~7.81 (7H, m), | | |
| | 7.87~7.94 (4H, m), 8.43 (1H, s), 8.55 (1H, m) | | |
| 8 | δ = 0.92 (12H, m), 1.78 (9H, m), 7.25 (1H, m), | 616.87 | 616.29 |
| | 7.32~7.38 (3H, m), 7.66~7.81 (6H, m), | | |
| | 7.87~7.96 (5H, m), 8.03 (1H, m), 8.1 (1H, m), | | |
| | 8.38 (1H, m), 8.55 (1H, m) | | |
| 9 | δ = 7.11 (1H, m), 7.25 (1H, m), 7.32 (1H, m), | 821.05 | 820.29 |
| | 7.33 (1H, m), 7.37~7.46 (22H, m), | | |
| | 7.64~7.81 (7H, m), 7.87~7.94 (3H, m), 8.27 (1H, | | |
| | m), 8.4 (1H, m), 8.55~8.6 (2H, m) | | |
| 10 | δ = 7.14 (1H, m), 7.25 (1H, m), 7.32~7.38 (5H, | 564.63 | 564.20 |
| | m), 7.66 (1H, m), 7.69 (1H, m), 7.7 (1H, m), | | |
| | 7.71 (1H, m), 7.72~7.81 (8H, m), 8.53~8.59 (3H, | | |
| | m), 9.3 (1H, m), 9.92 (1H, m) | | |
| 11 | δ = 7.14 (1H, m), 7.25 (2H, m), 7.32~7.41 (4H, | 563.65 | 563.20 |
| | m), 7.51~7.52 (4H, m), 7.66~7.81 (7H, m), | | |
| | 7.87~7.94 (3H, m), 8.53~8.55 (2H, m), 9.3 (1H, | | |
| | m), 9.41 (1H, m) | | |
| 12 | δ = 7.25 (1H, m), 7.32~7.41 (4H, m), | 562.66 | 562.20 |
| | 7.51~7.52 (4H, m), 7.66~7.81 (8H, m), | | |
| | 7.87~8 (4H, m), 8.3 (2H, m), 8.44 (1H, m), | | |
| | 8.55~8.6 (2H, m) | | |
| 13 | δ = 7.25 (1H, m), 7.32~7.41 (11H, m), | 638.75 | 638.24 |
| | 7.66~7.81 (8H, m), 7.87~7.94 (3H, m), 8.2 (2H, | | |
| | m), 8.3 (4H, m), 8.55 (1H, m) | | |
| 14 | δ = 7.25 (1H, m), 7.32~7.41 (11H, m), | 640.73 | 640.23 |
| | 7.66~7.81 (6H, m), 7.87~7.94 (3H, m), | | |
| | 8.26~8.3 (3H, m), 8.55~8.56 (2H, m), 9.93 (2H, | | |
| | m) | | |
| 15 | δ = 7.25 (1H, m), 7.32~7.51 (11H, m), | 640.73 | 640.23 |
| | 7.66~7.81 (6H, m), 7.87~7.94 (3H, m), 8.09 (1H, | | |
| | m), 8.28 (5H, m), 8.55 (1H, m) | | |
| 16 | δ = 7.25 (1H, m), 7.32~7.46 (16H, m), 7.55 (3H, | 821.05 | 820.29 |
| | m), 7.61 (1H, m), 7.62~7.71 (10H, m), | | |
| | 7.87~8 (4H, m), 8.3 (2H, m), 8.44 (1H, m), | | |
| | 8.55~8.6 (2H, m) | | |
| 17 | δ = 1.72 (6H, s), 7 (1H, m), 7.17 (1H, m), | 602.72 | 602.24 |
| | 7.25~7.26 (2H, m), 7.32~7.38 (4H, m), 7.51 (1H, | | |
| | m), 7.66~7.81 (6H, m), 7.87~7.96 (5H, m), | | |
| | 8.07 (1H, m), 8.14 (1H, m), 8.5~8.55 (2H, m) | | |
| 18 | δ = 7.32~7.4 (3H, m), 7.66~7.81 (8H, m), | 460.52 | 460.16 |
| | 7.87~8.01 (6H, m), 8.16 (1H, m), 8.41 (1H, m), | | |
| | 8.54 (1H, m) | | |
| 20 | δ = 7.26 (1H, m), 7.32~7.4 (4H, m), | 536.62 | 536.19 |
| | 7.47~7.55 (5H, m), 7.66~7.81 (8H, m), | | |
| | 7.87~7.89 (2H, m), 8.16 (2H, m), 8.3 (2H, m) | | |
| 21 | δ = 7.32~7.41 (3H, m), 7.51~7.52 (5H, m), | 536.62 | 536.19 |
| | 7.66~7.81 (8H, m), 7.87~7.96 (4H, m), 8.16 (1H, | | |
| | m), 8.4 (1H, m), 8.47 (1H, m), 8.54 (1H, m) | | |
| 22 | δ = 7.11 (1H, m), 7.32~7.54 (10H, m), | 612.72 | 612.22 |
| | 7.66~7.81 (8H, m), 7.87~7.96 (4H, m), 8.16 (1H, | | |
| | m), 8.3 (2H, m), 8.54 (1H, m), 8.6 (1H, m) | | |
| 25 | δ = 7.11 (1H, m), 7.25 (4H, m), 7.32~7.41 (4H, | 764.91 | 764.28 |
| | m), 7.51~7.52 (8H, m), 7.66~7.81 (8H, m), | | |
| | 7.87~7.96 (6H, m), 8.16 (1H, m), 8.54 (1H, m), | | |
| | 8.6 (1H, m), 8.81 (2H, m) | | |
| 31 | δ = 7.32~7.46 (15H, m), 7.55 (3H, m), 7.61 (1H, | 871.11 | 870.31 |
| | m), 7.62 (1H, m), 7.66~7.71 (11H, m), | | |
| | 7.87~8 (5H, m), 8.16 (1H, m), 8.3 (2H, m), | | |
| | 8.44 (1H, m), 8.54 (1H, m), 8.6 (1H, m) | | |
| 32 | δ = 7.32~7.54 (10H, m), 7.66~7.81 (8H, m), | 690.79 | 690.24 |
| | 7.87~7.96 (4H, m), 8.16 (1H, m), 8.26~8.3 (3H, | | |
| | m), 8.54~8.56 (2H, m), 9.93 (2H, m) | | |
| 33 | δ = 1.72 (6H, s), 7 (1H, m), 7.17 (1H, m), | 652.78 | 652.25 |
| | 7.26 (1H, m), 7.32~7.38 (3H, m), 7.51 (1H, m), | | |
| | 7.66~7.81 (8H, m), 7.87~7.96 (6H, m), 8.07 (1H, | | |
| | m), 8.14~8.16 (2H, m), 8.5~8.54 (2H, m) | | |
| 34 | δ = 7.32~7.51 (10H, m), 7.66~7.81 (8H, m), | 690.79 | 690.24 |
| | 7.87~7.96 (4H, m), 8.09 (1H, m), 8.16 (1H, m), | | |
| | 8.28 (5H, m), 8.54 (1H, m) | | |
| 35 | δ = 7.41~7.54 (18H, m), 7.69~7.81 (11H, m), | 790.95 | 790.30 |
| | 7.87 (1H, m), 8 (1H, m), 8.18~8.2 (3H, m), | | |
| | 8.3 (4H, m) | | |
| 36 | δ = 7.11 (1H, m), 7.22~7.25 (5H, m), | 715.84 | 715.26 |
| | 7.32~7.41 (4H, m), 7.51~7.52 (8H, m), | | |
| | 7.66~7.81 (5H, m), 7.88~7.89 (3H, m), | | |
| | 7.97~8 (2H, m), 8.18 (1H, m), 8.43 (1H, m), | | |
| | 8.6 (1H, m), 8.81 (2H, m) | | |
| 37 | δ = 7.11 (1H, m), 7.32~7.54 (11H, m), | 563.65 | 563.20 |
| | 7.66~7.81 (5H, m), 7.89 (1H, m), 8 (1H, m), | | |
| | 8.18 (1H, m), 8.3 (2H, m), 8.43 (1H, m), | | |
| | 8.6 (1H, m), 9.34 (1H, m) | | |
| 38 | δ = 7.11 (1H, m), 7.32~7.41 (11H, m), | 563.65 | 563.20 |
| | 7.66~7.81 (5H, m), 7.89 (1H, m), 8 (1H, m), | | |
| | 8.18 (1H, m), 8.3 (2H, m), 8.43 (1H, m), | | |
| | 8.51 (1H, m), 8.6 (1H, m) | | |
| 39 | δ = 7.11 (1H, m), 7.32 (1H, m), 7.38~7.47 (11H, | 613.70 | 613.22 |
| | m), 7.66~7.81 (6H, m), 7.89~7.92 (2H, m), | | |
| | 8 (1H, m), 8.18 (1H, m), 8.3 (2H, m), 8.6 (1H, | | |
| | m), 8.91 (1H, m) | | |
| 40 | δ = 7.11 (1H, m), 7.32~7.54 (10H, m), | 614.69 | 614.21 |
| | 7.66~7.81 (6H, m), 7.89 (1H, m), 8 (1H, m), | | |
| | 8.18 (1H, m), 8.3 (2H, m), 8.6 (1H, m), | | |
| | 8.75 (1H, m), 9.39 (2H, m) | | |
| 41 | δ = 7.11 (1H, m), 7.25~7.33 (4H, m), | 779.88 | 779.27 |
| | 7.41~7.54 (10H, m), 7.63 (1H, m), | | |
| | 7.66~7.71 (8H, m), 7.87 (1H, m), 7.94 (1H, m), | | |
| | 8.12 (1H, m), 8.3 (2H, m), 8.55~8.6 (2H, m), | | |
| | 8.74 (2H, m) | | |
| 42 | δ = 7.25 (1H, m), 7.32~7.41 (5H, m), 7.51 (4H, | 563.65 | 563.20 |
| | m), 7.66~7.81 (10H, m), 7.87~7.94 (3H, m), | | |
| | 8.55 (1H, m), 8.63 (1H, s), (H,) | | |
| 43 | δ = 7.25 (1H, m), 7.32~7.41 (5H, m), 7.51 (4H, | 564.63 | 564.20 |
| | m), 7.66~7.81 (6H, m), 7.87~7.94 (3H, m), | | |
| | 8.28 (4H, m), 8.55 (1H, m) | | |
| 44 | δ = 7.25 (1H, m), 7.32 (2H, s), 7.32~7.41 (4H, | 563.65 | 563.20 |
| | m), 7.51 (4H, m), 7.66~7.81 (8H, m), | | |
| | 7.87~7.94 (3H, m), 8.28 (2H, m), 8.55 (1H, m) | | |
| 45 | δ = 1.72 (12H, s), 7.25~7.38 (8H, m), 7.55 (2H, | 795.97 | 795.32 |
| | m), 7.63~7.81 (10H, m), 7.87~7.94 (7H, m), | | |
| | 8.55 (1H, m), 8.63 (1H, s), (H,) | | |
| 46 | δ = 7.25 (1H, m), 7.33 (1H, m), 7.41 (2H, m), | 580.70 | 580.17 |
| | 7.5~7.52 (6H, m), 7.69 (1H, m), 7.77 (1H, m), | | |
| | 7.86~7.87 (2H, m), 7.94~8 (4H, m), 8.28 (4H, | | |
| | m), 8.45 (1H, m), 8.55 (1H, m) | | |
| 47 | δ = 7.25 (1H, m), 7.33 (1H, m), 7.41 (2H, m), | 731.90 | 731.24 |
| | 7.5~7.52 (10H, m), 7.69 (1H, m), 7.77 (1H, m), | | |
| | 7.85~7.87 (6H, m), 7.94~8 (4H, m), 8.3 (4H, m), | | |
| | 8.45 (1H, m), 8.55 (1H, m), 8.63 (1H, s), (H,) | | |
| 48 | δ = 7.11 (1H, m), 7.25 (1H, m), 7.33 (1H, m), | 578.72 | 578.18 |
| | 7.41~7.54 (10H, m), 7.69 (1H, m), 7.77 (1H, m), | | |
| | 7.86~7.87 (2H, m), 7.94~8 (4H, m), 8.3 (2H, m), | | |
| | 8.45 (1H, m), 8.55~8.6 (2H, m) | | |
| 49 | δ = 7.25 (1H, m), 7.33 (1H, m), 7.41 (2H, m), | 579.71 | 579.18 |
| | 7.5~7.52 (6H, m), 7.69 (1H, m), 7.77~7.79 (5H, | | |
| | m), 7.86~7.87 (2H, m), 7.94~8 (4H, m), | | |
| | 8.45 (1H, m), 8.55 (1H, m), 8.63 (1H, s), (H,) | | |
| 50 | δ = 1.72 (6H, s), 7.25~7.41 (6H, m), | 589.73 | 589.25 |
| | 7.51~7.55 (6H, m), 7.61 (1H, m), 7.69 (1H, m), | | |
| | 7.77~7.79 (5H, m), 7.87 (2H, m), 7.94 (1H, m), | | |
| | 8.06 (1H, m), 8.55 (1H, m), 8.63 (1H, s), (H,) | | |
| 51 | δ = 1.72 (6H, s), 7.25~7.41 (6H, m), | 590.71 | 590.25 |
| | 7.51~7.55 (6H, m), 7.61 (1H, m), 7.69 (1H, m), | | |
| | 7.77 (1H, m), 7.87 (2H, m), 7.94 (1H, m), | | |
| | 8.06 (1H, m), 8.28 (4H, m), 8.55 (1H, m) | | |
| 52 | δ = 1.72 (6H, s), 7.11 (1H, m), 7.25 (1H, m), | 588.74 | 588.26 |
| | 7.28 (1H, m), 7.33 (1H, m), 7.38~7.51 (11H, m), | | |
| | 7.61 (1H, m), 7.69 (1H, m), 7.77 (1H, m), | | |
| | 7.87 (2H, m), 7.94 (1H, m), 8.06 (1H, m), | | |
| | 8.3 (2H, m), 8.55~8.6 (2H, m) | | |
| 53 | δ = 1.72 (6H, s), 7.25~7.41 (6H, m), | 741.92 | 741.31 |
| | 7.48~7.61 (15H, m), 7.69~7.77 (6H, m), | | |
| | 7.87 (2H, m), 7.94 (1H, m), 8.06 (1H, m), | | |
| | 8.55 (1H, m), 8.63 (1H, s), (H,) | | |
| 54 | δ = 0.66 (6H, s), 7.25 (1H, m), 7.33 (2H, m), | 605.80 | 605.23 |
| | 7.41 (2H, m), 7.51~7.52 (5H, m), 7.58~7.61 (2H, | | |
| | m), 7.69 (1H, m), 7.77~7.94 (10H, m), 8.55 (1H, | | |
| | m), 8.63 (1H, s), (H,) | | |
| 55 | δ = 1.3 (4H, m), 1.45 (4H, m), 7.25 (1H, m), | 632.83 | 632.24 |
| | 7.33 (2H, m), 7.41 (2H, m), 7.51~7.52 (5H, m), | | |
| | 7.58~7.61 (2H, m), 7.69 (1H, m), 7.77~7.94 (6H, | | |
| | m), 8.28 (4H, m), 8.55 (1H, m) | | |
| 56 | δ = 7.25 (1H, m), 7.33~7.42 (6H, m), | 655.72 | 655.22 |
| | 7.48~7.51 (5H, m), 7.69~7.87 (12H, m), | | |
| | 7.94 (1H, m), 8.03~8.12 (3H, m), 8.55 (1H, m), | | |
| | 8.63 (1H, s), (H,) | | |
| 57 | δ = 7.25 (1H, m), 7.33~7.55 (26H, m), 7.69 (1H, | 893.95 | 893.34 |
| | m), 7.72~7.79 (13H, m), 7.94 (1H, m), 8.55 (1H, | | |
| | m), 8.63 (1H, s), (H,) | | |
| 58 | δ = 7.25 (1H, m), 7.33 (1H, m), 7.41~7.51 (11H, | 635.56 | 635.25 |
| | m), 7.69~7.81 (11H, m), 7.87~7.88 (3H, m), | | |
| | 7.94 (1H, m), 8.55 (1H, m), 8.63 (1H, s), (H,) | | |
| 59 | δ = 7.25 (1H, m), 7.33 (1H, m), 7.41~7.51 (11H, | 671.72 | 671.21 |
| | m), 7.69 (1H, m), 7.75 (1H, m), 7.77~7.87 (13H, | | |
| | m), 8.55 (1H, m), 8.63 (1H, s), (H,) | | |
| 60 | δ = 7.25~7.33 (3H, m), 7.41~7.51 (10H, m), | 638.76 | 638.25 |
| | 7.58~7.63 (3H, m), 7.69 (1H, m), 7.77~7.79 (6H, | | |
| | m), 7.87 (1H, m), 7.94~8 (2H, m), 8.12 (1H, m), | | |
| | 8.18 (1H, m), 8.55 (1H, m), 8.63 (1H, s), (H,) | | |
| 61 | δ = 7.25 (2H, m), 7.33 (2H, m), 7.41~7.51 (9H, | 639.75 | 639.24 |
| | m), 7.58 (2H, m), 7.69 (2H, m), 7.77 (2H, m), | | |
| | 7.87 (2H, m), 7.94 (2H, m), 8.28 (4H, m), | | |
| | 8.55 (2H, m) | | |
| 62 | δ = 7.25 (2H, m), 7.33 (2H, m), 7.41~7.51 (11H, | 715.84 | 715.27 |
| | m), 7.58 (2H, m), 7.69 (2H, m), 7.77 (2H, m), | | |
| | 7.87 (2H, m), 7.94 (2H, m), 8.09 (1H, m), | | |
| | 8.28 (5H, m), 8.55 (2H, m) | | |
| 63 | δ = 7.25 (1H, m), 7.33~7.51 (11H, m), 7.58 (2H, | 640.73 | 640.24 |
| | m), 7.69 (1H, m), 7.77 (2H, m), 7.87 (1H, m), | | |
| | 7.94~8 (2H, m), 8.18 (1H, m), 8.28 (4H, m), | | |
| | 8.43 (1H, m), 8.51~8.55 (2H, m) | | |
| 64 | δ = 7.25 (1H, m), 7.33 (1H, m), 7.41~7.51 (11H, | 690.79 | 690.25 |
| | m), 7.58 (2H, m), 7.69 (1H, m), 7.76~7.77 (3H, | | |
| | m), 7.87~8 (4H, m), 8.18 (1H, m), 8.28 (4H, m), | | |
| | 8.55 (1H, m), 8.91 (1H, m) | | |
| 65 | δ = 7.25 (1H, m), 7.33 (1H, m), 7.41~7.56 (8H, | 579.71 | 579.18 |
| | m), 7.69 (1H, m), 7.77~7.79 (3H, m), | | |
| | 7.86~7.87 (2H, m), 7.94~8 (4H, m), 8.09 (1H, | | |
| | m), 8.28 (1H, m), 8.45 (1H, m), 8.54~8.55 (2H, | | |
| | m) | | |
| 66 | δ = 7.25~7.33 (3H, m), 7.41 (2H, m), | 643.79 | 643.28 |
| | 7.5~7.51 (5H, m), 7.63 (1H, m), 7.69 (1H, m), | | |
| | 7.77~7.79 (6H, m), 7.87 (1H, m), 7.94~8 (2H, | | |
| | m), 8.12 (1H, m), 8.18 (1H, m), 8.55 (1H, m), | | |
| | 8.63 (1H, s), (H,) | | |
| 67 | δ = 7.25~7.33 (3H, m), 7.41 (4H, m), | 793.91 | 793.30 |
| | 7.5~7.51 (9H, m), 7.63 (1H, m), 7.69 (1H, m), | | |
| | 7.77~7.79 (6H, m), 7.87 (1H, m), 7.94~8 (2H, | | |
| | m), 8.12 (1H, m), 8.18 (1H, m), 8.28 (4H, m), | | |
| | 8.55 (1H, m), 8.63 (1H, s), (H,) | | |
| 68 | δ = 7.25 (1H, m), 7.33~7.42 (26H, m), | 1024.29 | 1023.38 |
| | 7.61~7.69 (7H, m), 7.76~7.77 (3H, m), 7.87 (1H, | | |
| | m), 7.94~8 (2H, m), 8.09 (1H, m), | | |
| | 8.16~8.18 (2H, m), 8.28 (4H, m), 8.54~8.55 (2H, | | |
| | m) | | |

### Example 1

### Manufacture of OLED Device Using the Organic Electroluminescent Compound According to the Present Invention

An OLED device was manufactured using the electroluminescent material according to the present invention. First, a transparent electrode ITO thin film (15Ω/□) obtained from a glass for OLED (produced by Samsung Corning) was subjected to ultrasonic washing with trichloroethylene, acetone, ethanol and distilled water, sequentially, and stored in isopropanol before use.

Then, an ITO substrate was equipped in a substrate folder of a vacuum vapor deposition apparatus, and 4,4′,4″-tris(N,N-(2-naphthyl)-phenylamino)triphenylamine (2-TNATA) was placed in a cell of the vacuum vapor deposition apparatus, which was then ventilated up to 10⁻⁶ torr of vacuum in the chamber. Then, electric current was applied to the cell to evaporate 2-TNATA, thereby forming a hole injection layer having a thickness of 60 nm on the ITO substrate.

Then, N,N′-bis(α-naphthyl)-N,N′-diphenyl-4,4′-diamine (NPB) was placed in another cell of the vacuum vapor deposition apparatus, and electric current was applied to the cell to evaporate NPB, thereby forming a hole transport layer having a thickness of 20 nm on the hole injection layer.

After forming the hole injection layer and the hole transport layer, an electroluminescent layer was formed thereon as follows. Compound 49 was placed in a cell of a vacuum vapor deposition apparatus as host, and Ir(ppy)₃[tris(2-phenylpyridine)iridium] was placed in another cell as a dopant. The two materials were evaporated at different rates such that an electroluminescent layer having a thickness of 30 nm was vapor-deposited on the hole transport layer at 4 to 10 wt %.

Subsequently, tris(8-hydroxyquinoline)-aluminum(III) (Alq) was vapor-deposited with a thickness of 20 nm as an electron transport layer. Then, after vapor-depositing lithium quinolate (Liq) of a following structure with a thickness of 1 to 2 nm as an electron injection layer, an Al cathode having a thickness of 150 nm was formed using another vacuum vapor deposition apparatus to manufacture an OLED. Each compound used in the OLED was purified by vacuum sublimation at 10⁻⁶ torr.

### Example 2

### Manufacture of OLED Device Using the Organic Electroluminescent Compounds of the Present Invention

An OLED was manufactured as in Example 1 except that Compound 23 according to the present invention is used as host material on the electroluminescent layer and an organic iridium complex (piq)₂Ir(acac)[bis-(1-phenylisoquinolyl)iridium(III)acetylacetonate] is used as electroluminescent dopant.

### Comparative Examples 1 and 2

### Electroluminescent Properties of the OLED Device Using Conventional Electroluminescent Material

An OLED device was manufactured in the same manner as Examples 1 and 3 except that 4,4′-Bis(carbazol-9-yl)-biphenyl (CBP) instead of the compounds of the present invention was used as host material in a cell of the vacuum vapor deposition apparatus.

Luminous efficiency of the OLED devices including the organic electroluminescent compound according to the present invention manufactured in Examples 1 to 2 and Comparative Examples 1 and 2 and the conventional electroluminescent compound was measured at 1,000 cd/m². The result is given in Table 2.

TABLE 2

| | | | | Hole | Driving | Power | |
|---|---|---|---|---|---|---|---|
| | | | | blocking | voltage(V) | efficiency(cd/A) | |
| No. | | Host | Dopant | layer | @1,000 cd/m² | @1,000 cd/m² | Color |
| Example | 6 | 49 | Ir(ppy)₃ | — | 6.7 | 30.1 | Green |
| 1 | 7 | 59 | Ir(ppy)₃ | — | 6.5 | 28.1 | Green |
| | 8 | 60 | Ir(ppy)₃ | — | 6.7 | 30.3 | Green |
| | 9 | 62 | Ir(ppy)₃ | — | 6.4 | 28.3 | Green |
| | 10 | 67 | Ir(ppy)₃ | — | 6.3 | 29.0 | Green |
| Example | 16 | 23 | (piq)₂Ir(acac) | — | 6.2 | 7.1 | Red |
| 2 | 17 | 30 | (piq)₂Ir(acac) | — | 6.1 | 7.6 | Red |
| | 18 | 34 | (piq)₂Ir(acac) | — | 6.0 | 7.8 | Red |
| | 19 | 53 | (piq)₂Ir(acac) | — | 6.3 | 7.6 | Red |
| | 20 | 55 | (piq)₂Ir(acac) | — | 6.4 | 7.3 | Red |

| | | | | | | |
|---|---|---|---|---|---|---|
| Comparative | CBP | Ir(ppy)₃ | BAlq | 7.5 | 25.1 | Green |
| Example 1 | | | | | | |
| Comparative | CBP | (piq)₂Ir(acac) | BAlq | 7.5 | 6.5 | Red |
| Example 2 | | | | | | |

As shown in Table 2, the organic electroluminescent compounds according to the present invention have excellent properties compared with the conventional material. In addition, the device using the organic electroluminescent compound according to the present invention as host material for emitting red or green light has excellent electroluminescent properties and drops driving voltage, thereby increasing power efficiency and improving power consumption.

What is claimed is:

1. An organic electroluminescent compound represented by Chemical Formula 1: 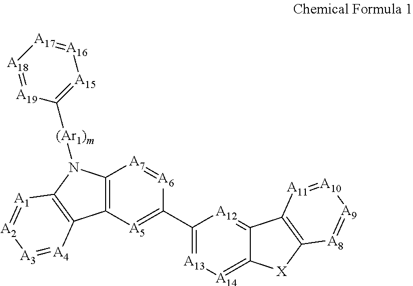
wherein
A₁ through A₁₉ independently represent CR₁ or N, X represents —(CR₂R₃)₁—, —N(R₄)—, —S—, —O—, —Si(R₅)(R₆)—, —P(R₇)—, —P(═O) (R₈)— or —B(R₉)—, and Ar₁ represents (C6-C40)arylene with or without substituent(s) or (C3-C40)heteroarylene with or without substituent(s), except for the case where m is 0, and A₁₅ through A₁₉ are CR₁ at the same time;
R₁ through R₉ independently represent hydrogen, deuterium, halogen, (C1-C30)alkyl with or without substituent(s), (C6-C30)aryl with or without substituent(s), substituted or unsubstituted (C6-C30)aryl fused with one or more (C3-C30)cycloalkyl(s) with or without substituent(s), (C3-C30)heteroaryl with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), cyano, trifluoromethyl, NR₂₁R₂₂, BR₂₃R₂₄, PR₂₅R₂₆, P(═O)R₂₇R₂₈, R^{a}R^{b}R^{c}Si—, R^{d}Y—, R^{e}C(═O)—, R^{f}C(═O)O—, (C6-C30)ar(C1-C30)alkyl with or without substituent(s), (C2-C30)alkenyl with or without substituent(s), (C2-C30)alkynyl with or without substituent(s), carboxyl, nitro, 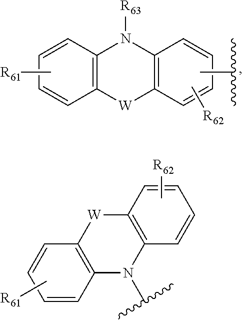
or hydroxyl, or each of them may be linked to an adjacent substituent via (C3-C30)alkylene or (C3-C30)alkenylene with or without a fused ring to form an alicylic ring, a mono- or polycyclic aromatic ring or a mono- or polycyclic heteroaromatic ring;
W represents —(CR₅₁R₅₂)ₙ—, —(R₅₁)C═C(R₅₂)—, —N(R₅₃)—, —S—, —O—, —Si (R₅₄)(R₅₅)—, —P(R₅₆)—, —P(═O)(R₅₇)—, —C(═O)— or —B(R₅₈)—, and R₅₁ through R₅₈ and R₆₁ through R₆₃ are the same as R₁ through R₉;
the heterocycloalkyl or heteroaryl may contain one or more heteroatom(s) selected from B, N, O, S, P(═O), Si and P;
R₂₁ through R₂₈ independently represent (C1-C30)alkyl with or without substituent(s), (C6-C30)aryl with or without substituent(s) or (C3-C30)heteroaryl with or without substituent(s), R^{a}, R^{b}, and R^{c} independently represent (C1-C30)alkyl with or without substituent(s) or (C6-C30)aryl with or without substituent(s), Y represents S or O, R^{d} represent (C1-C30)alkyl with or without substituent(s) or (C6-C30)aryl with or without substituent(s), R^{e} represent (C1-C30)alkyl with or without substituent(s), (C1-C30)alkoxy with or without substituent(s), (C6-C30)aryl with or without substituent(s) or (C6-C30)aryloxy with or without substituent(s), R^{f} represent (C1-C30)alkyl with or without substituent(s), (C1-C30)alkoxy with or without substituent(s), (C6-C30)aryl with or without substituent(s) or (C6-C30)aryloxy with or without substituent(s);
m represents an integer 0 to 2; and
l and n represent an integer 1 or 2.

2. The organic electroluminescent compound according to claim 1, wherein the substituent of R₁ through R₉, R₂₁ through R₂₉, R₅₁ through R₅₈ and R₆₁ through R₆₃ is further substituted by one or more substituent(s) selected from a group consisting of deuterium, halogen, (C1-C30)alkyl with or without halogen substituent(s), (C6-C30)aryl, (C3-C30)heteroaryl with or without (C6-C30)aryl substituent(s), 5- to 7-membered heterocycloalkyl, 5- to 7-membered heterocycloalkyl fused with one or more aromatic ring(s), (C3-C30)cycloalkyl, (C3-C30)cycloalkyl fused with one or more aromatic ring(s), tri(C1-C30)alkylsilyl, di(C1-C30)alkyl(C6-C30)arylsilyl, tri(C6-C30)arylsilyl, adamantyl, (C7-C30)bicycloalkyl, (C2-C30)alkenyl, (C2-C30)alkynyl, cyano, carbazolyl, NR₃₁R₃₂, BR₃₃R₃₄, PR₃₅R₃₆, P(═O)R₃₇R₃₈, (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro and hydroxyl, or is linked to an adjacent substituent to form a ring, wherein R₃₁ through R₃₈ independently represent (C1-C30)alkyl, (C6-C30)aryl or (C3-C30)heteroaryl.

3. The organic electroluminescent compound according to claim 1, wherein R₁ through R₉ are selected from the following structures: 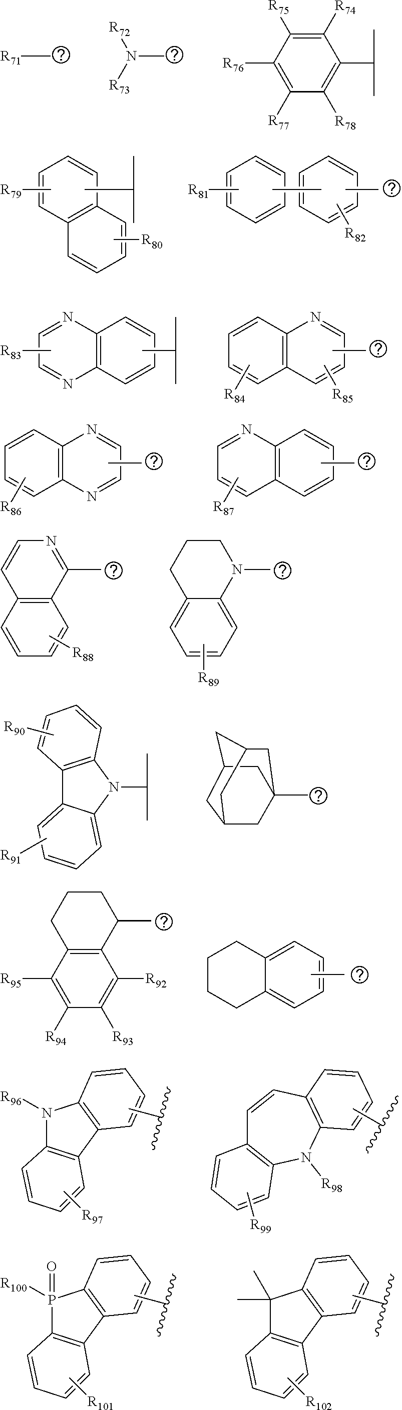 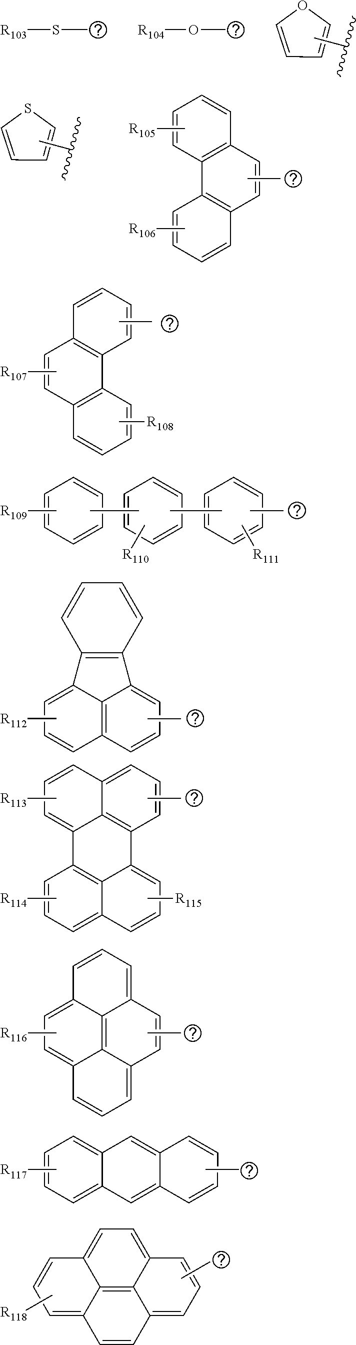 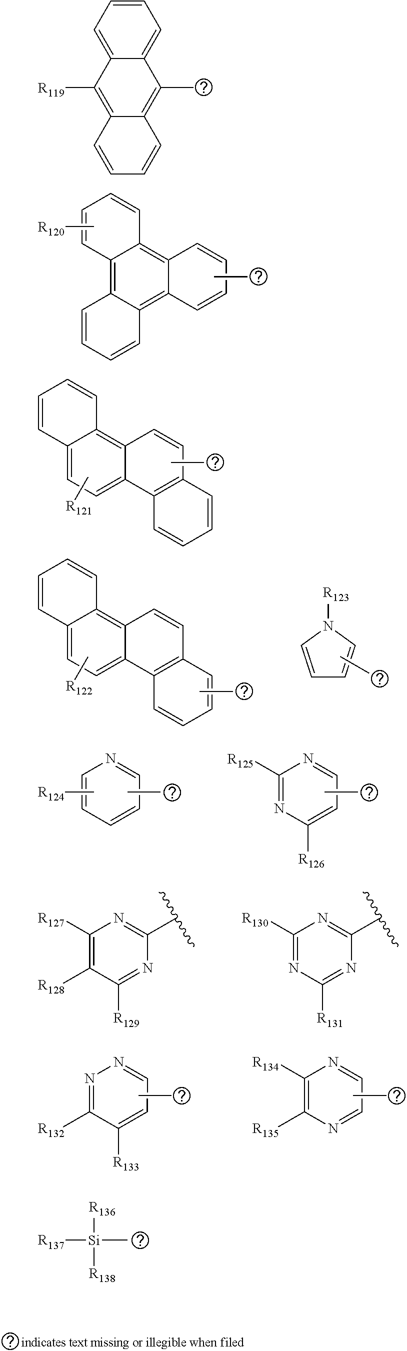
wherein
R₇₁ through R₁₃₈ independently represent hydrogen, deuterium, halogen, (C1-C30)alkyl, (C6-C30)aryl, (C6-C30)aryl fused with one or more (C3-C30)cycloalkyl(s), (C3-C30)heteroaryl, 5- to 7-membered heterocycloalkyl, 5- to 7-membered heterocycloalkyl fused with one or more aromatic ring(s), (C3-C30)cycloalkyl, (C3-C30)cycloalkyl fused with one or more aromatic ring(s), cyano, amino, (C1-C30)alkylamino, (C6-C30)arylamino, NR₄₁R₄₂, BR₄₃R₄₄, PR₄₅R₄₆, P(═O)R₄₇R₄₈, tri(C1-C30)alkylsilyl, di(C1-C30)alkyl(C6-C30)arylsilyl, tri(C6-C30)arylsilyl, (C6-C30)ar(C1-C30)alkyl, (C1-C30)alkyloxy, (C1-C30)alkylthio, (C6-C30)aryloxy, (C6-C30)arylthio, (C1-C30)alkoxycarbonyl, (C1-C30)alkylcarbonyl, (C6-C30)arylcarbonyl, (C2-C30)alkenyl, (C2-C30)alkynyl, (C6-C30)aryloxycarbonyl, (C1-C30)alkoxycarbonyloxy, (C1-C30)alkylcarbonyloxy, (C6-C30)arylcarbonyloxy, (C6-C30)aryloxycarbonyloxy, carboxyl, nitro or hydroxyl, or each of them may be linked to an adjacent substituent via (C3-C30)alkylene or (C3-C30)alkenylene with or without a fused ring to form an alicylic ring or a mono- or polycyclic aromatic ring, wherein R₄₁ through R₄₈ independently represent (C1-C30)alkyl, (C6-C30)aryl or (C3-C30)heteroaryl.

4. The organic electroluminescent compound according to claim 1, wherein 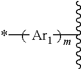
is selected from the following structures:

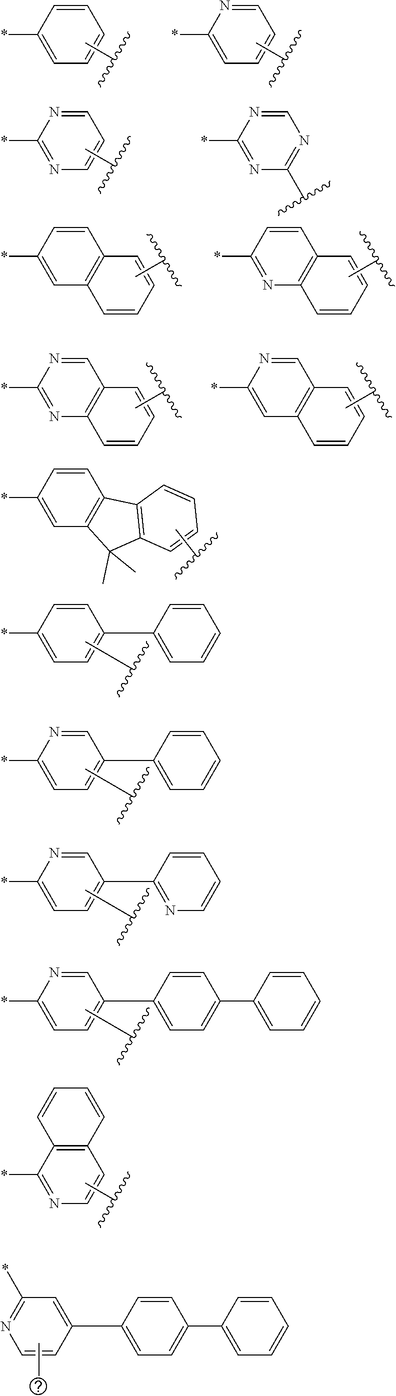

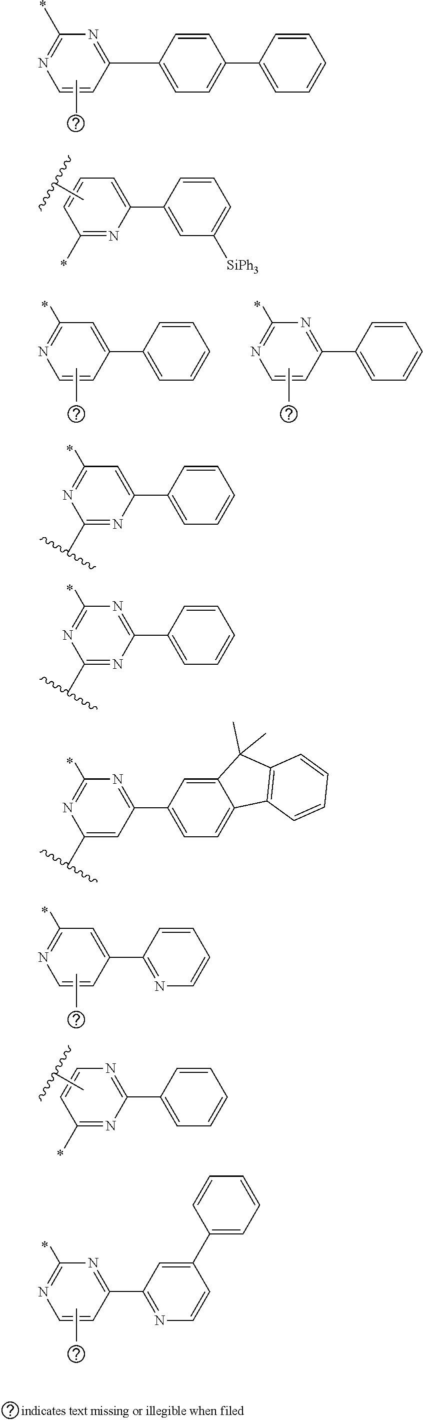

5. The organic electroluminescent compound according to claim 1, which is selected from the following structures:

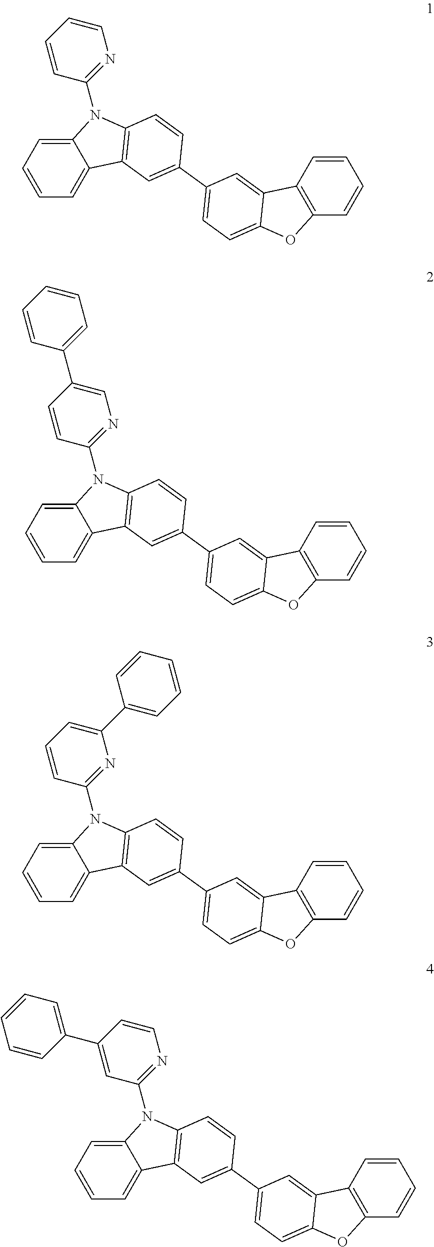

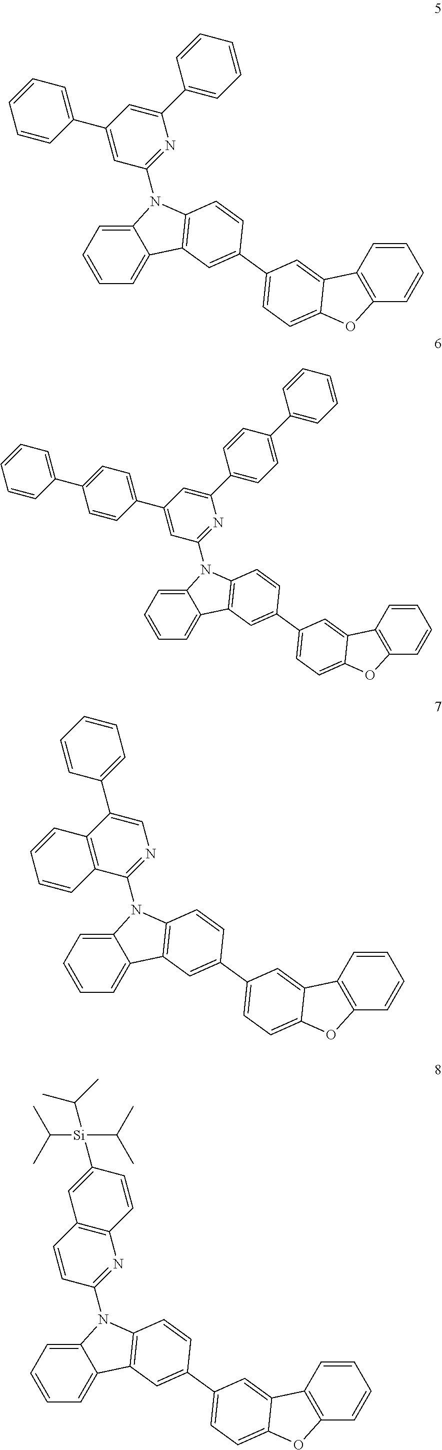

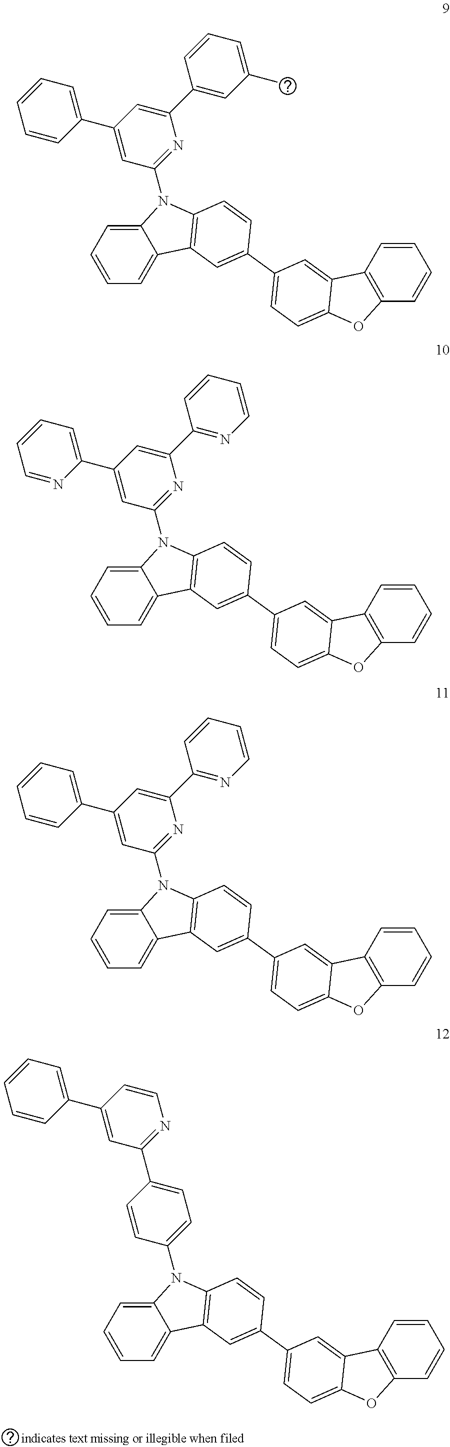

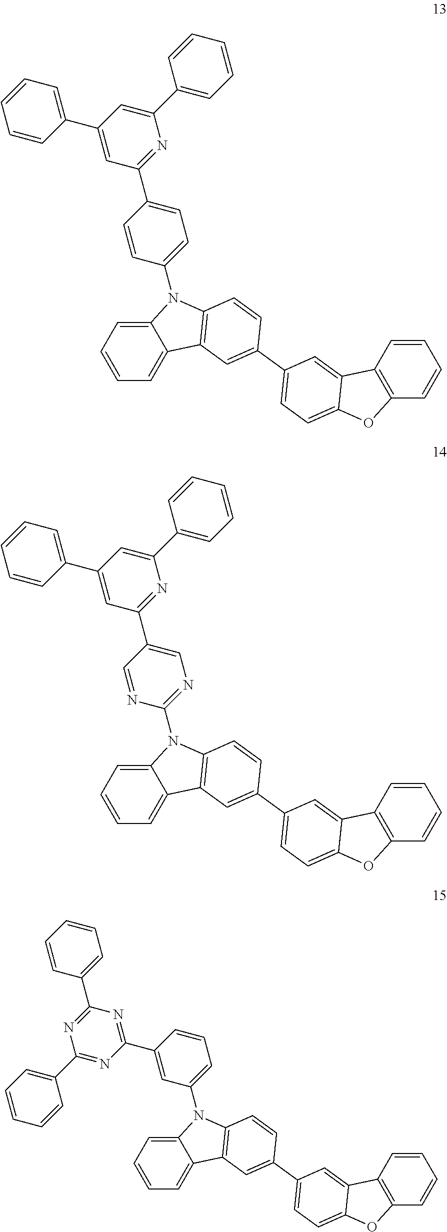

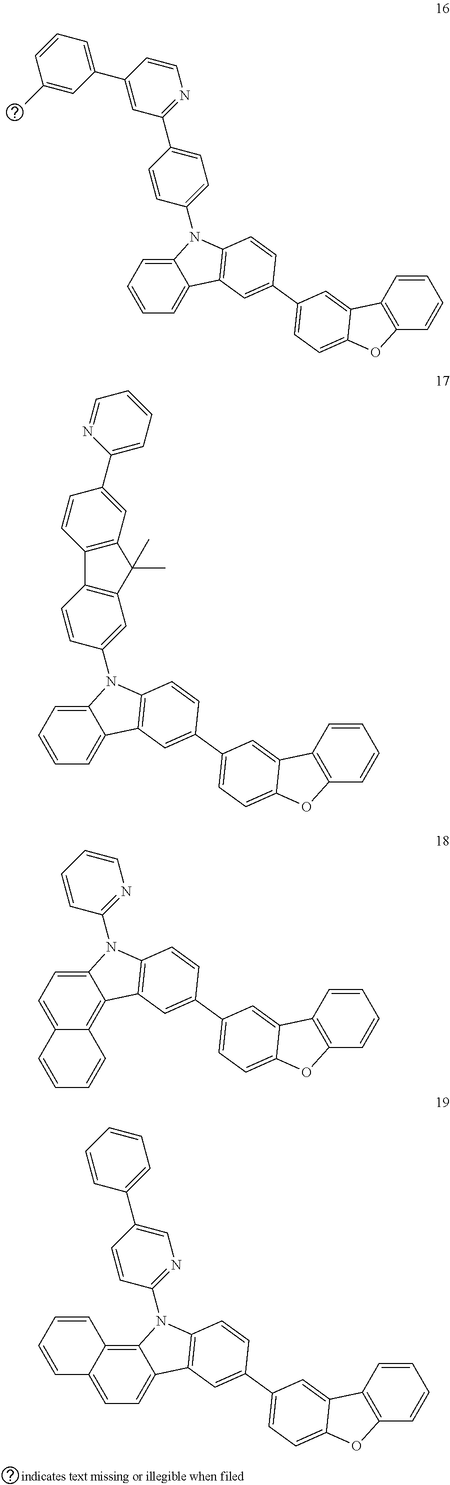

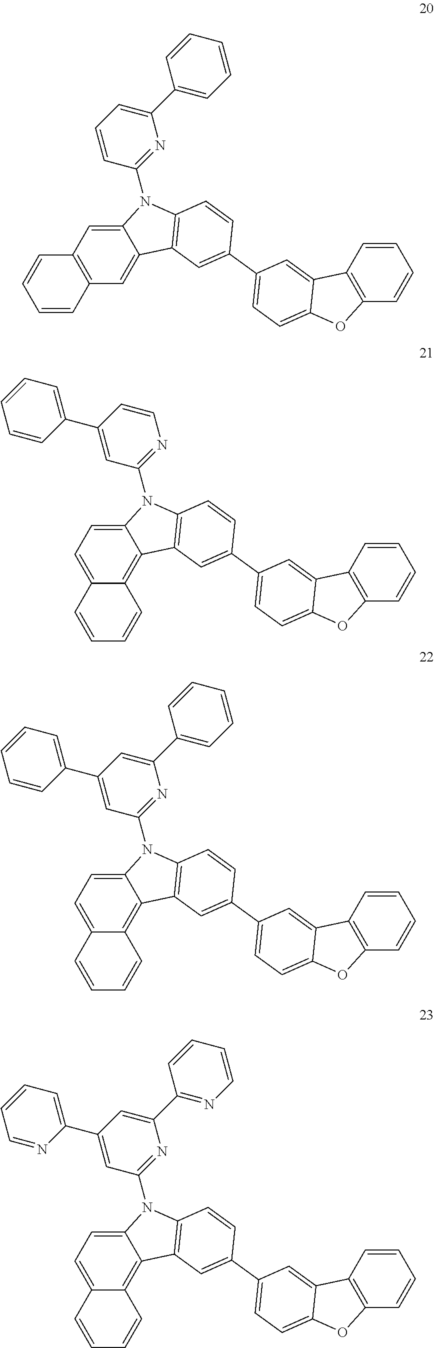

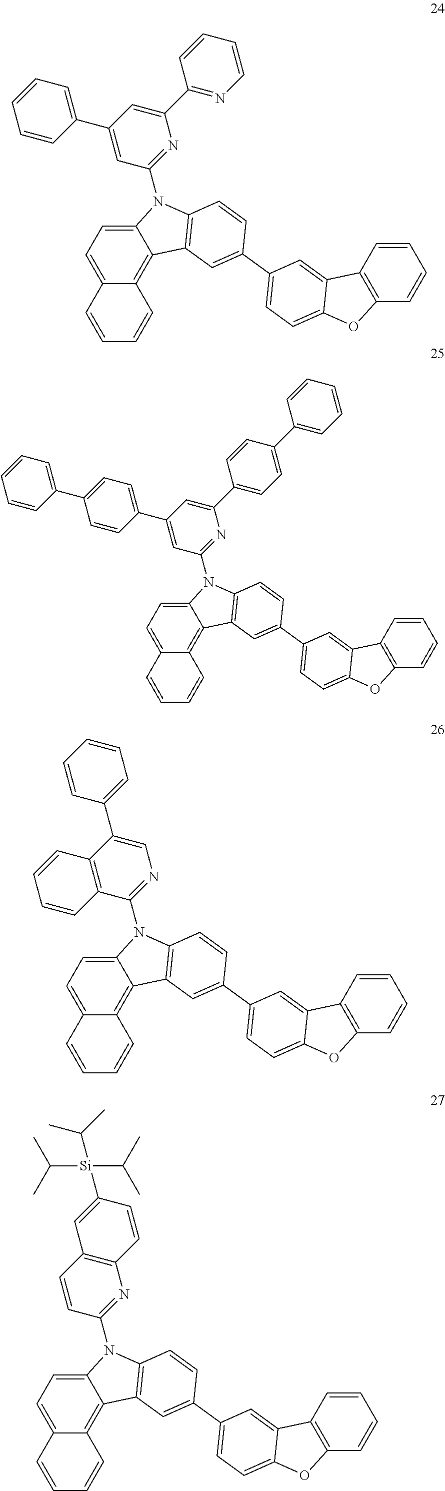

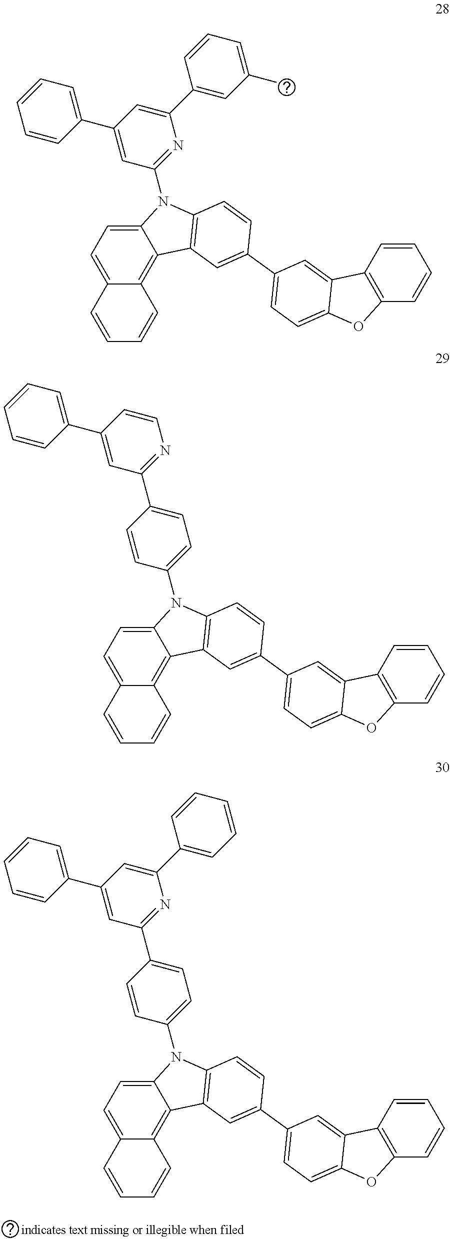

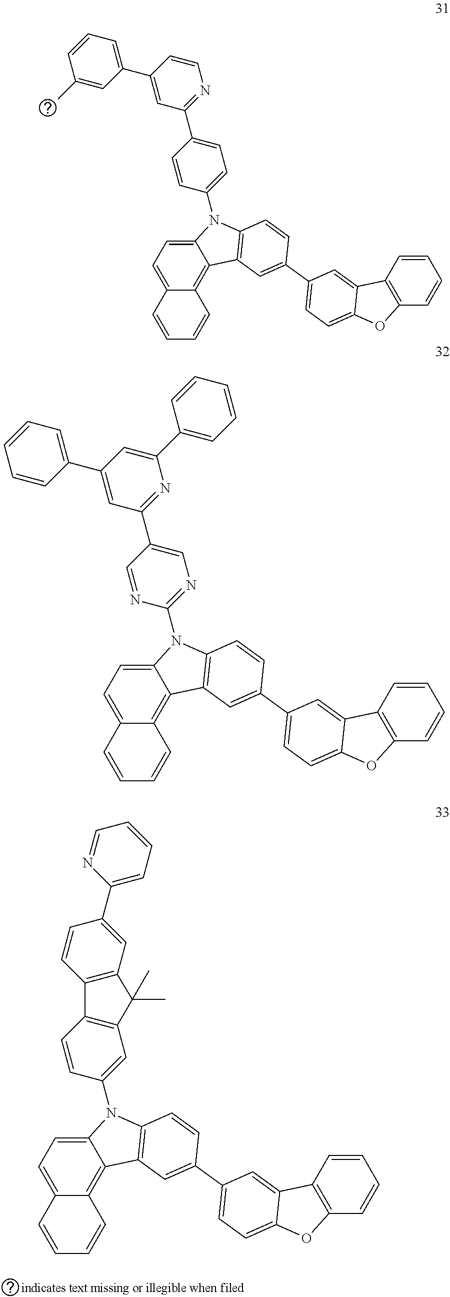

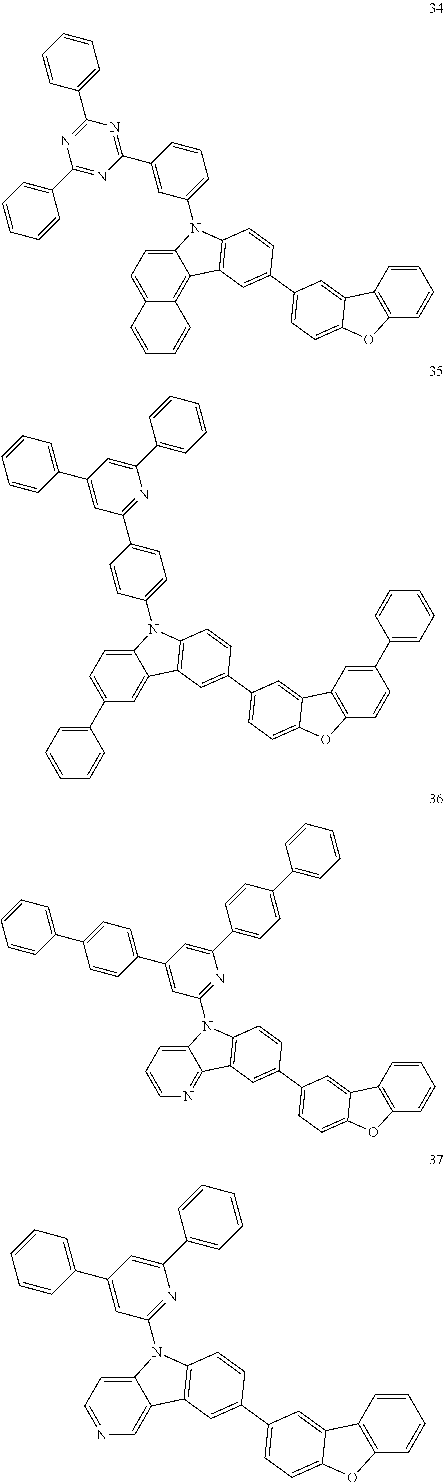

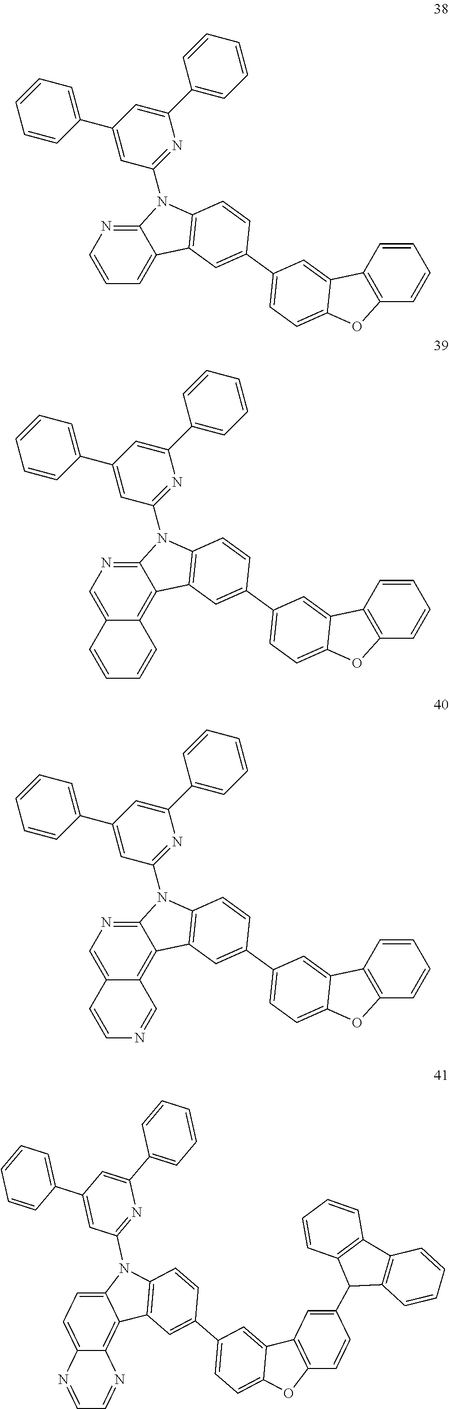

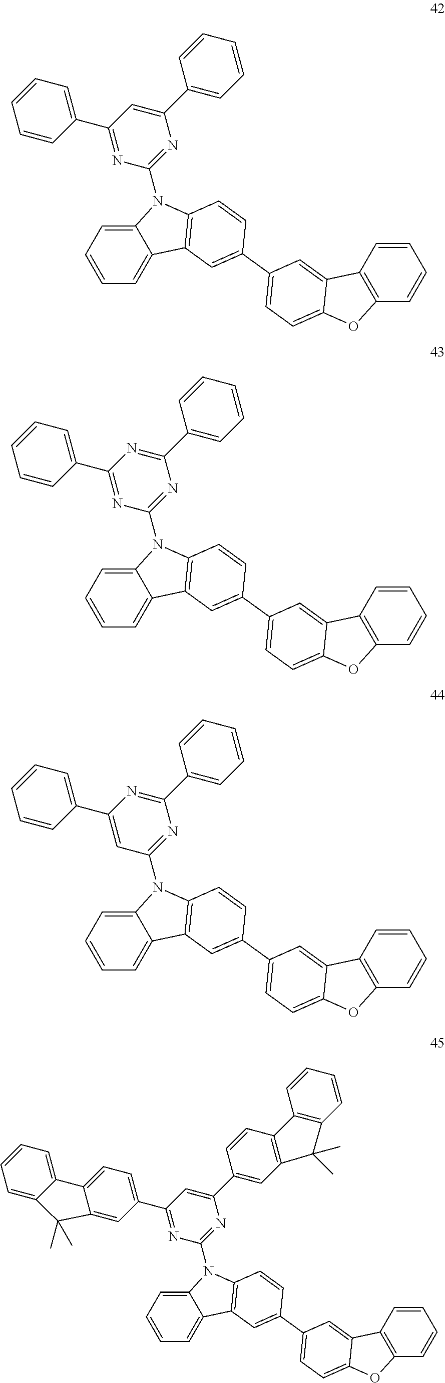

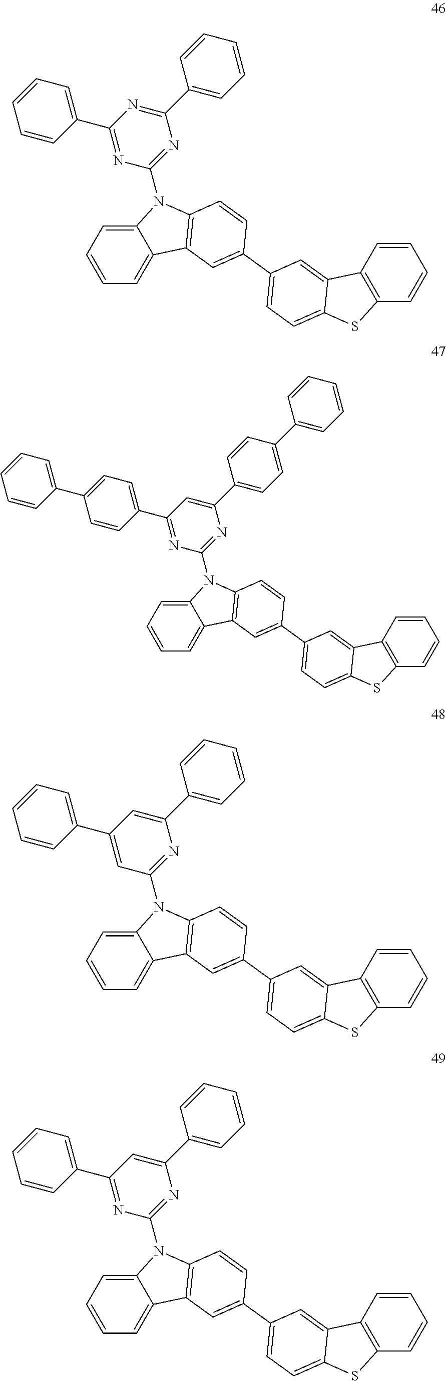

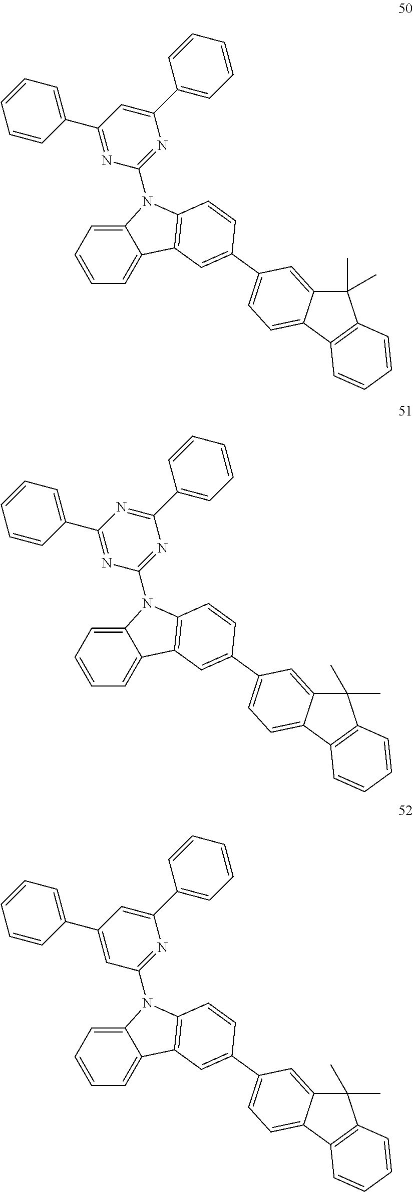

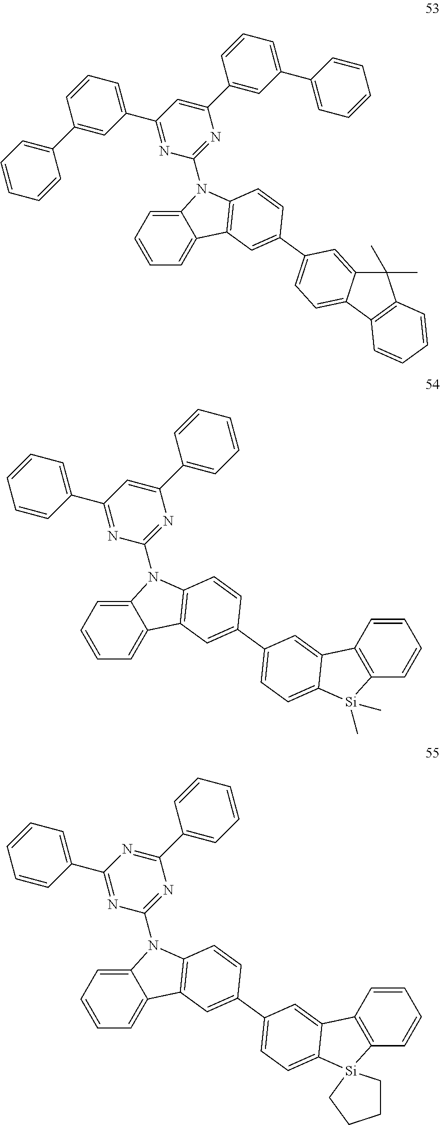

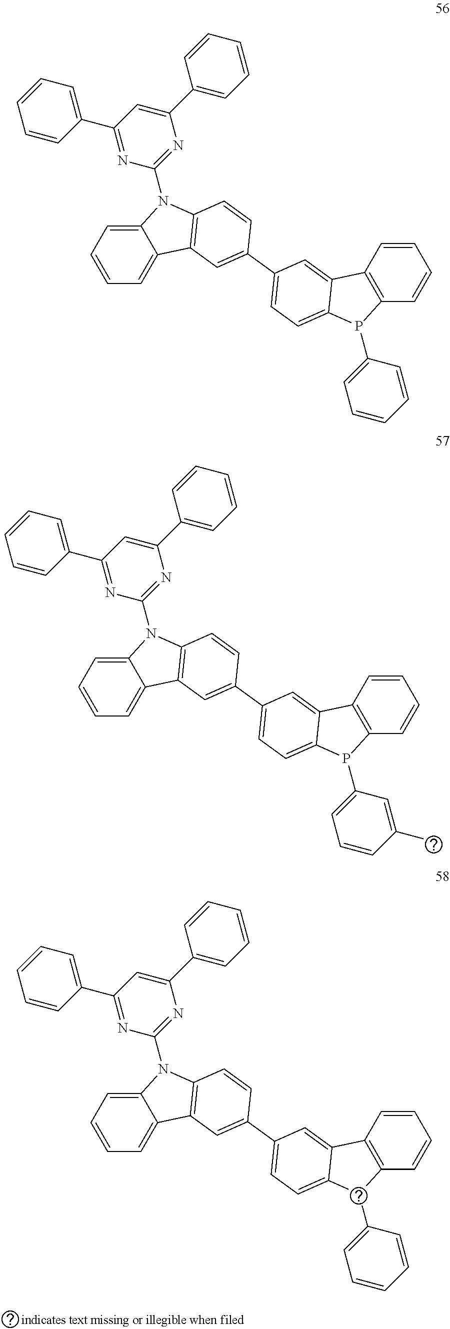

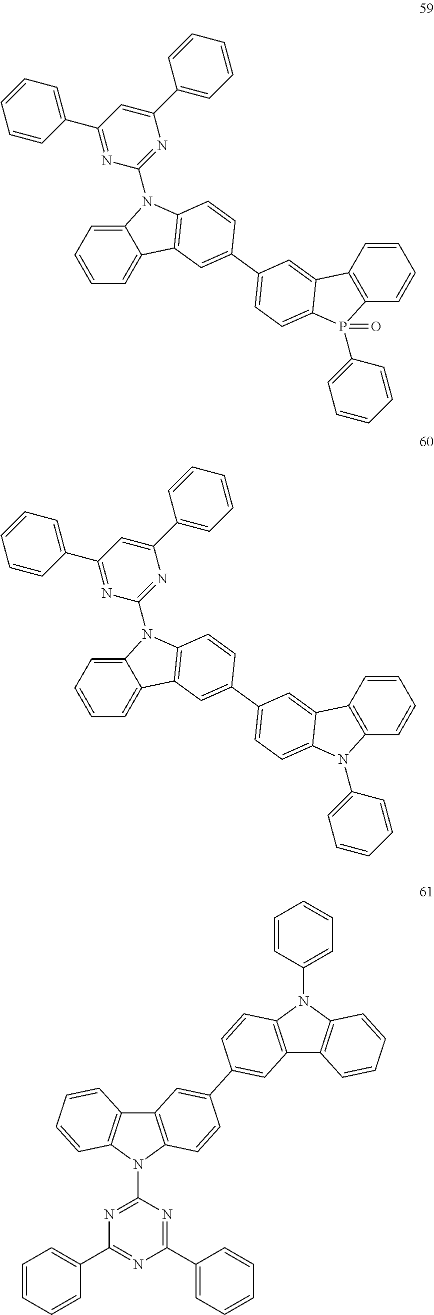

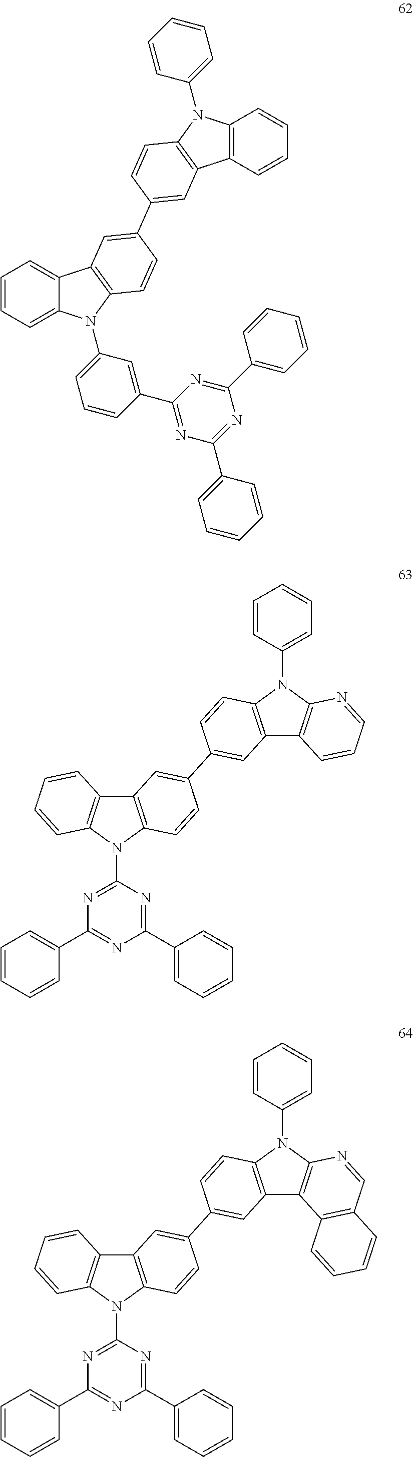

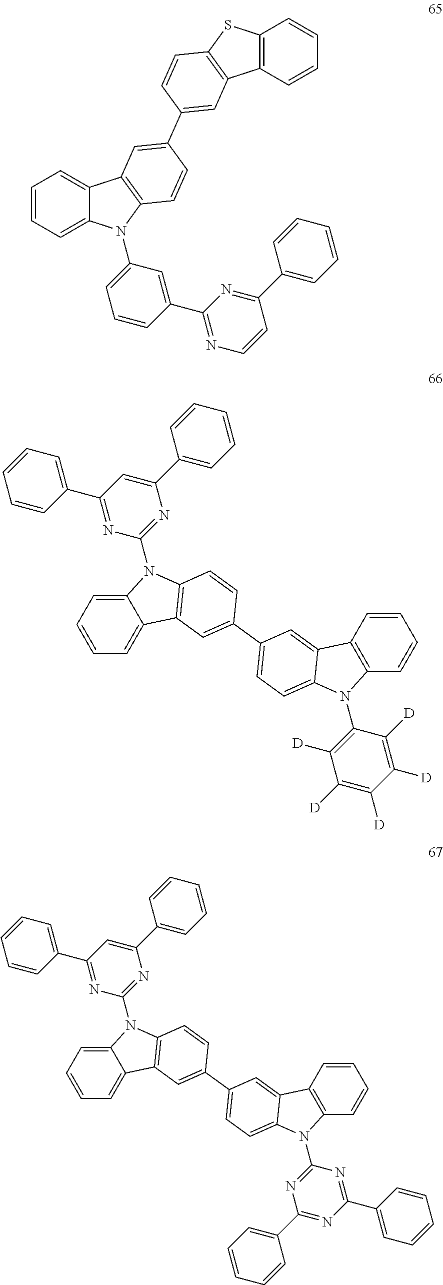

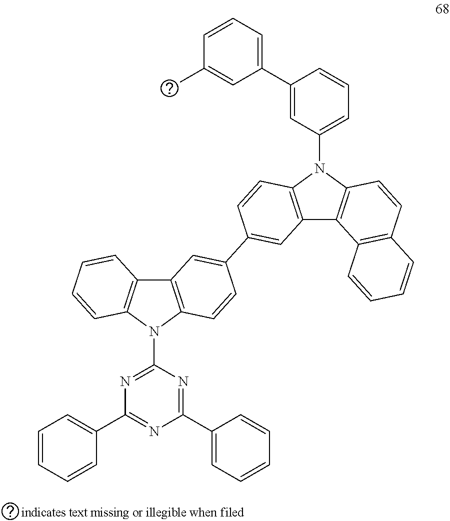

6. An organic electroluminescent device comprising the organic electroluminescent compound according any of claims 1 to 5.

7. The organic electroluminescent device according to claim 6, which comprises a first electrode; a second electrode; and one or more organic layer(s) interposed between the first electrode and the second electrode, wherein the organic layer comprises one or more organic electroluminescent compound(s) according to any of claims 1 to 5 and one or more dopant(s) represented by Chemical Formula 2:
M¹L¹⁰¹L¹⁰²L¹⁰³ Chemical Formula 2
wherein
M¹ is a metal selected from a group consisting of Group 7, Group 8, Group 9, Group 10, Group 11, Group 13, Group 14, Group 15 and Group 16 metals, and ligand L¹⁰¹, L¹⁰² and L¹⁰³ are independently selected from the following structures: 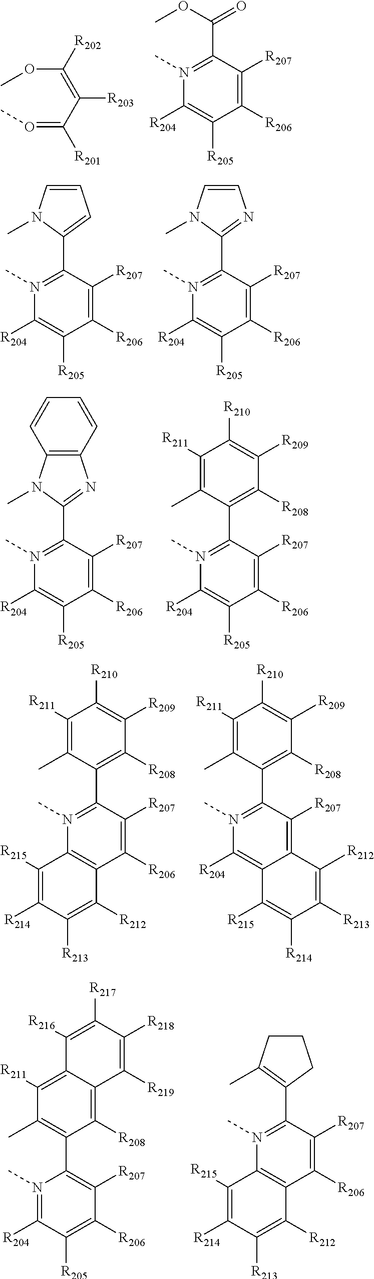 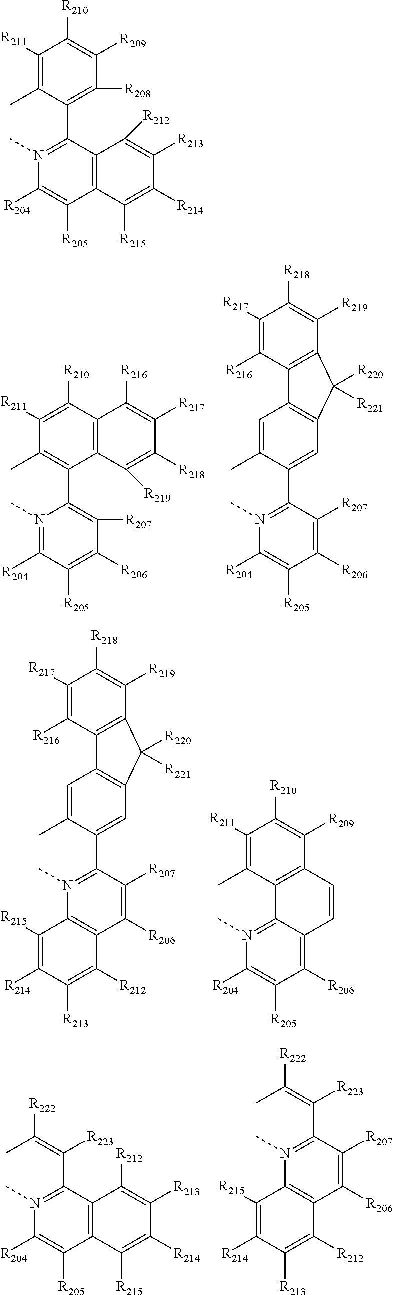 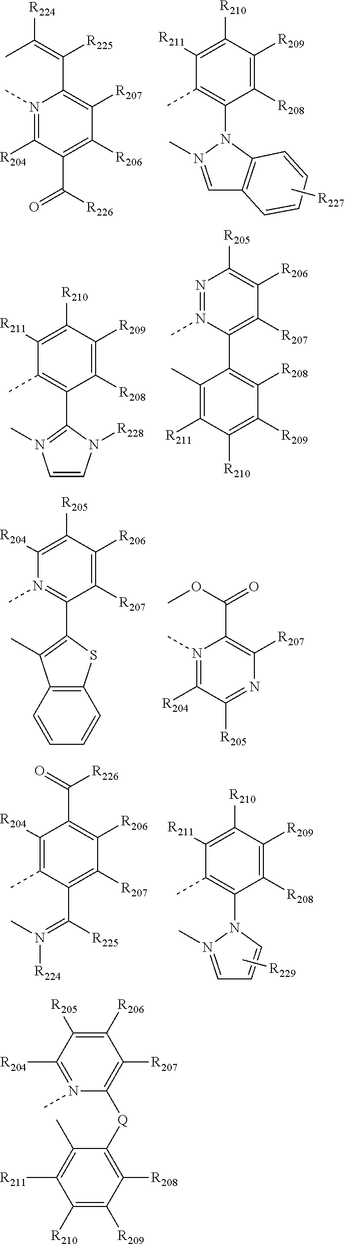
wherein
R₂₀₁ through R₂₀₃ independently represent hydrogen, (C1-C30)alkyl with or without halogen substituent(s), (C6-C30)aryl with or without (C1-C30)alkyl substituent(s) or halogen;
R₂₀₄ through R₂₁₉ independently represent hydrogen, (C1-C30)alkyl with or without substituent(s), (C1-C30)alkoxy with or without substituent(s), (C3-C30)cycloalkyl with or without substituent(s), (C2-C30)alkenyl with or without substituent(s), (C6-C30)aryl with or without substituent(s), mono- or di-(C1-C30)alkylamino with or without substituent(s), mono- or di-(C6-C30)arylamino with or without substituent(s), SF₅, tri(C1-C30)alkylsilyl with or without substituent(s), di(C1-C30)alkyl(C6-C30)arylsilyl with or without substituent(s), tri(C6-C30)arylsilyl with or without substituent(s), cyano or halogen;
R₂₂₀ through R₂₂₃ independently represent hydrogen, (C1-C30)alkyl with or without halogen substituent(s) or (C6-C30)aryl with or without (C1-C30)alkyl substituent(s);
R₂₂₄ and R₂₂₅ independently represent hydrogen, (C1-C30)alkyl with or without substituent(s), (C6-C30)aryl with or without substituent(s) or halogen, or R₂₂₄ and R₂₂₅ may be linked via (C3-C12)alkylene or (C3-C12)alkenylene with or without a fused ring to form an alicylic ring or a mono- or polycyclic aromatic ring;
R₂₂₆ represents (C1-C30)alkyl with or without substituent(s), (C6-C30)aryl with or without substituent(s), (C5-C30)heteroaryl with or without substituent(s) or halogen;
R₂₂₇ through R₂₂₉ independently represent hydrogen, (C1-C30)alkyl with or without substituent(s), (C6-C30)aryl with or without substituent(s) or halogen; and
Q represents 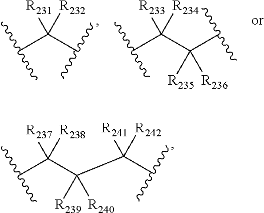
wherein R₂₃₁ through R₂₄₂ independently represent hydrogen, (C1-C30)alkyl with or without halogen substituent(s), (C1-C30)alkoxy, halogen, (C6-C30)aryl with or without substituent(s), cyano or (C5-C30)cycloalkyl with or without substituent(s), or each of them may be linked to an adjacent substituent via alkylene or alkenylene to form a spiro ring or a fused ring, or may be linked to R₂₀₇ or R₂₀₈ via alkylene or alkenylene to form a saturated or unsaturated fused ring.

8. The organic electroluminescent device according to claim 7, wherein the organic layer further comprises one or more amine compound(s) selected from a group consisting of arylamine compounds and styrylarylamine compounds, or one or more metal(s) selected from a group consisting of organic metals of Group 1, Group 2, 4th period and 5th period transition metals, lanthanide metals and d-transition elements or complex compound(s).

9. The organic electroluminescent device according to claim 7, wherein the organic layer comprises an electroluminescent layer and a charge generating layer.

10. The organic electroluminescent device according to claim 7, which is a white light-emitting organic electroluminescent device wherein the organic layer comprises one or more organic compound layer(s) emitting red, green or blue light at the same time.
